(19) United States
(12) Patent Application Publication
(10) Pub. No.: US 2023/0381490 A1
(43) Pub. Date: Nov. 30, 2023

(54) SURGICAL SYSTEM FOR MANAGING BLOOD FLOW DURING A SURGICAL PROCEDURE

(71) Applicant: Excision Medical, Inc., Malvern, PA (US)

(72) Inventors: Greg Alan WALTERS, Malvern, PA (US); Francois JOUIN, Malvern, PA (US); Alison Mikayla KANE, Malvern, PA (US); Michael A. DOTSEY, Malvern, PA (US); Chad J. SMITH, Malvern, PA (US); Jason KEISER, Malvern, PA (US)

(73) Assignee: Excision Medical, Inc., Malvern, PA (US)

(21) Appl. No.: 18/202,346

(22) Filed: May 26, 2023

Related U.S. Application Data
(60) Provisional application No. 63/346,029, filed on May 26, 2022.

(51) Int. Cl. A61M 60/35; A61M 60/13; A61M 60/268; A61M 60/837; A61M 60/865; A61M 60/139; A61F 2/24; A61F 2/01

(52) U.S. Cl. CPC A61M 60/35; A61M 60/13; A61M 60/268; A61M 60/837; A61M 60/865; A61M 60/139; A61F 2/2427; A61F 2/014; A61F 2/0105

(57) ABSTRACT

The present disclosure describes a surgical system for a valve in an aorta. The surgical system is configured to serve as a temporary valve and/or filter to facilitate maintenance of proper blood flow while also capturing debris of an excised leaflet as needed.

### CROSS-REFERENCE TO RELATED APPLICATIONS

The present application claims the benefit of and right of priority to U.S. Provisional Application No. 63/346,029, filed May 26, 2022, the entire contents of which are incorporated by reference into the present application.

### TECHNICAL FIELD

The present disclosure relates to a surgical system for maintaining proper blood flow during a surgical procedure.

### BACKGROUND

Transcatheter aortic valve replacement (TAVR) is an alternative option for the treatment of patients with severe calcific aortic stenosis. Indeed, TAVR may become the preferred therapy for all patients irrespective of surgical risk. However, transcatheter heart valves (THV) may fail in the future and repeat intervention may be required. So-called redo-transcatheter aortic valve implantation (TAVI) or TAVR may lead to risks of coronary obstruction due to the leaflet of the failed valve being pushed up by the new valve and leading to obstruction of blood flow to the coronary arteries. TAVR in failed surgical bioprostheses is common. However, TAVR in failed transcatheter bioprostheses (i.e., transcatheter heart valve-in-transcatheter heart valve) will also become increasingly common. In both situations there is a risk of coronary obstruction. The risk of coronary obstruction can be predicted with the use of cardiac computed tomography. If the predicted risk of coronary occlusion is high, then percutaneous valve-in-valve intervention may be prohibitive. In some cases, the cause of the coronary obstruction is related to the leaflets of the failed surgical or transcatheter heart valve that are pushed up and prevent flow of blood to the coronary arteries.

### SUMMARY

There is a need for systems, devices, and procedures for maintaining proper blood flow during excision of portions of an aortic valve. As such, an embodiment of the present disclosure is a surgical system with a shaft that is elongated along a central axis. The surgical system further includes an expandable shield carried by the shaft. The expandable shield has an insertion configuration, where the expandable shield is collapsed toward the central axis. The expandable shield also has an expanded configuration, where the expandable shield is expanded outwardly away from the central axis. The surgical system further includes an actuatable panel coupled to the expandable shield. The actuatable panel is configured to transition between A) a closed configuration that inhibits fluid flow through the expandable shield in both a first direction and a second direction that is opposite the first direction, and B) an open configuration that permits fluid flow through the expandable shield in both the first direction and the second direction. The surgical system further includes an actuator coupled to the actuatable panel and configured to cause the actuatable panel to transition between the closed configuration and the open configuration.

Another embodiment is a surgical system that includes a shaft that is elongated along a central axis. The surgical system further includes an expandable shield carried by the shaft. The expandable shield includes a non-porous section, a porous section, an insertion configuration, where the expandable shield is collapsed toward the central axis, and an expanded configuration, where the expandable shield is expanded outwardly away from the central axis. The surgical system includes a movable panel on the expandable shield. The movable panel is configured to open in response to fluid flow in a first direction that impinges the expandable shield, and close in response to fluid flow in a second direction that is opposite the first direction that impinges the expandable shield. The surgical system further includes an actuatable panel that overlies the porous section of the expandable shield. The actuatable panel is configured to transition between A) a closed configuration that inhibits fluid flow through the expandable shield in both the first direction and the second direction, and B) an open configuration that permits fluid flow through the expandable shield in both the first direction and the second direction. The surgical system further includes an actuator coupled to the actuatable panel and configured to cause the actuatable panel to transition between the closed configuration and the open configuration.

Another embodiment of the present disclosure is a surgical system including a shaft that is elongated along a central axis. The surgical system further includes an expandable shield carried by the shaft. The expandable shield has an insertion configuration, where the expandable shield is collapsed toward the central axis, and an expanded configuration, where the expandable shield is expanded outwardly away from the central axis. The surgical system further includes a movable panel coupled to the expandable shield. The movable panel is configured to open in response to a fluid flow in a first direction that impinges the expandable shield, and close in response to fluid flow in a second direction that is opposite the first direction that impinges the expandable shield.

Another embodiment of the present disclosure is a method. The method includes inserting an expandable shield into an aorta in an insertion configuration along a guidewire to a location in the ascending aorta. The method further includes expanding the expandable shield from the insertion configuration into an expanded configuration such that an outer perimeter of the expandable shield is positioned outwardly toward an aortic wall. The method further includes actuating an actuatable panel overlying a porous section of the expandable shield, between an open configuration and a closed configuration to selectively permit blood flow through the porous section of the expandable shield.

Another embodiment of the present disclosure is a method. The method includes inserting an expandable shield into an aorta in an insertion configuration along a guidewire to a location in the ascending aorta. The method further includes expanding the expandable shield from the insertion configuration into an expanded configuration such that an outer perimeter of the expandable shield is positioned outwardly toward an aortic wall. The method further includes permitting the movable panel to open and close in response to systolic and diastolic blood flow. The method further includes actuating an actuatable panel overlying a porous section of the expandable shield, between an open configuration and a closed configuration to selectively permit blood flow through the porous section of the expandable shield.

### BRIEF DESCRIPTION OF THE DRAWINGS

The foregoing summary, as well as the following detailed description of illustrative embodiments of the present application, will be better understood when read in conjunction with the appended drawings. For purposes of illustrating the present application, the drawings show exemplary embodiments of the present disclosure. It should be understood, however, that the present disclosure is not limited to the precise arrangements and instrumentalities shown in the drawings. In the drawings:

FIG. 1 is a side view of a surgical system according to an embodiment of the present disclosure;

FIG. 2 is an exploded side view of the surgical system illustrated in FIG. 1;

FIG. 3 is a side view of the surgical system shown in FIG. 1, with the expandable shield inside the catheter;

FIG. 4 is a side view of the surgical system shown in FIG. 3, with the expandable shield in an insertion configuration;

FIG. 5 is a side view of the surgical system shown in FIGS. 3-4, with the expandable shield in an expanded configuration;

FIGS. 6 and 7 illustrate an expandable shield frame of the surgical system shown in FIGS. 1-5 in an insertion configuration and an expanded configuration, respectively, according to an embodiment of the present disclosure;

FIGS. 8 and 9 illustrate an expandable shield frame of the surgical system shown in FIGS. 1-5 in an insertion configuration and an expanded configuration, respectively, according to an embodiment of the present disclosure;

FIGS. 10 and 11 illustrate an expandable shield frame of the surgical system shown in FIGS. 1-5 in an insertion configuration and an expanded configuration, respectively, according to an embodiment of the present disclosure;

FIG. 12 is a detailed view of a distal end of the surgical system shown in FIGS. 1-5, with the movable/actuatable panel shown in a closed configuration;

FIGS. 13A and 13B is a detailed view of a distal end of the surgical system shown in FIG. 12, with the movable/actuatable panel shown in an open configuration;

FIG. 14A is a detailed view of a distal end of the surgical system shown in FIGS. 1-5, with the movable/actuatable panel shown in a closed configuration, according to another embodiment of the disclosure;

FIGS. 14B and 14C is a detailed view of a distal end of the surgical system shown in FIG. 14A, with the movable/actuatable panel shown in an open configuration;

FIG. 15 illustrates the expandable shield shown in FIGS. 12-14C with a tapered panel as blood flows in a first (F1) direction;

FIG. 16 illustrates the expandable shield shown in FIG. 15 as blood flows in a second (F2) direction;

FIG. 17 illustrates the expandable shield shown in FIGS. 12-14C with a curved panel as blood flows in a first direction;

FIG. 18 illustrates the expandable shield shown in FIG. 17 as blood flows in a second direction;

FIG. 19 illustrates the expandable shield shown in FIGS. 12-14C with a slitted panel as blood flows in a first direction;

FIG. 20 illustrates the expandable shield shown in FIG. 19 as blood flows in a second direction;

FIG. 21 illustrates the expandable shield with a distensible outer panel as blood flows in a first direction;

FIG. 22 illustrates the expandable shield shown in FIG. 21 as blood flows in a second direction;

FIG. 23 illustrates the expandable shield shown in FIGS. 12-14C with a deflected configuration as blood flows in a first direction;

FIG. 24 illustrates the expandable shield shown in FIG. 23 with an expanded configuration as blood flows in a second direction;

FIG. 25A is a side schematic view of a heart showing placement of various sheaths and guidewires in a cardiovascular system, including a TAVR sheath with a wire (LV wire) extending into the left ventricle and another sheath with another wire (AA wire) extending into the ascending aorta;

FIG. 25B illustrates a dilator and/or introducer placed over the LV wire and advanced into the ascending aorta and showing a steerable catheter advanced over the dilator/introducer;

FIG. 25C illustrates the steerable catheter steered into position and the dilator/introducer removed;

FIG. 25D illustrates a hemoshield catheter advanced over the AA wire;

FIG. 25E illustrates deployment of the hemoshield distal to the valve;

FIG. 25F illustrates deployment of the hemoshield distal to the valve and expanded outwardly and adjustment of regurgitation as needed;

FIG. 25G illustrates deployment of the surgical system through the steerable catheter;

FIG. 25H illustrates adjustment of the catheter so that the distal end of the surgical system is in contact with the leaflet of the valve;

FIG. 25I illustrates a piercing element puncturing the leaflet of the valve, using radio frequency energy from an electrosurgical unit;

FIG. 25J illustrates a capture element advancing into a position and seizing the leaflet;

FIG. 25K illustrates the first cutting element advanced to the leaflet and forming a curved opening while the capture element seizes the leaflet;

FIG. 25L illustrates the second cutting element (first and second cutting hooks) advancing through the curved opening while the capture element seizes the leaflet;

FIG. 25M illustrates the surgical system being retracted slightly so the first and second cutting hooks are able to splay apart while applying tension to the cutters to ensure contact with the leaflet;

FIG. 25N illustrates cutting the leaflet portion from the valve;

FIG. 25O illustrates cutting the leaflet portion from the valve;

FIG. 25P illustrates the surgical system retracting the cutters, capture element and excised portion of leaflet into the catheter;

FIG. 25Q illustrates readjustment of the regurgitation via the hemoshield as needed and retraction of the surgical system into the steering catheter; the above illustrated steps can be repeated for each valve;

FIG. 26 illustrates the surgical system having a braided shield, according to another embodiment of the present disclosure;

FIGS. 27 and 28 illustrates the expandable shield shown in FIG. 26 where the movable panel is coupled to the internal side of the expandable shield as blood flows in a first direction and second direction;

FIGS. 29 and 30 illustrates the expandable shield shown in FIG. 26 where the movable panel is coupled to the external side of the expandable shield as blood flows in a first direction and second direction; and

FIGS. 31A through 31N illustrate the surgical system shown in FIGS. 26-30, deployed in a cardiovascular system, and performing a surgical procedure while maintaining and controlling blood flow.

FIG. 31A illustrates the surgical system shown in FIGS. 26-30, deployed in a cardiovascular system;

FIG. 31B illustrates the surgical system shown in FIGS. 26-30, with the expandable shield exiting the sheath;

FIG. 31C illustrates the surgical system shown in FIGS. 26-30, deployed in a cardiovascular system, with the expandable shield fully deployed and inhibiting flow;

FIG. 31D illustrates the surgical system shown in FIGS. 26-30, deployed in a cardiovascular system, with the expandable shield fully deployed and movable panels moving in response to fluid flow;

FIG. 31E illustrates the surgical system shown in FIGS. 26-30, deployed in a cardiovascular system, with a wire inserted through the expandable shield;

FIG. 31F illustrates the surgical system shown in FIGS. 26-30, deployed in a cardiovascular system, with a surgical device inserted through the expandable shield for performing a surgical procedure;

FIG. 31G illustrates the surgical system shown in FIGS. 26-30, deployed in a cardiovascular system, with a surgical device removed from the expandable shield;

FIGS. 31H and 31I illustrate the surgical system shown in FIGS. 26-30, deployed in a cardiovascular system, showing retraction of the expandable shield into the sheath;

FIG. 31J illustrates the surgical system deployed in a cardiovascular system, according to another embodiment of the present disclosure, where shield is configured for inversion to capture debris;

FIGS. 31K, 31L and 31M illustrate the surgical system shown in FIGS. 31J, showing retracting of a shaft and leading end of the shield inwardly;

FIG. 31N illustrates the surgical system shown in FIG. 31J with the shield fully retracted into the sheath.

### DETAILED DESCRIPTION OF ILLUSTRATIVE EMBODIMENTS

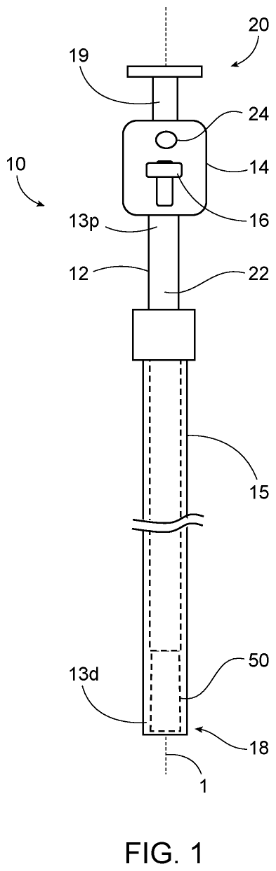

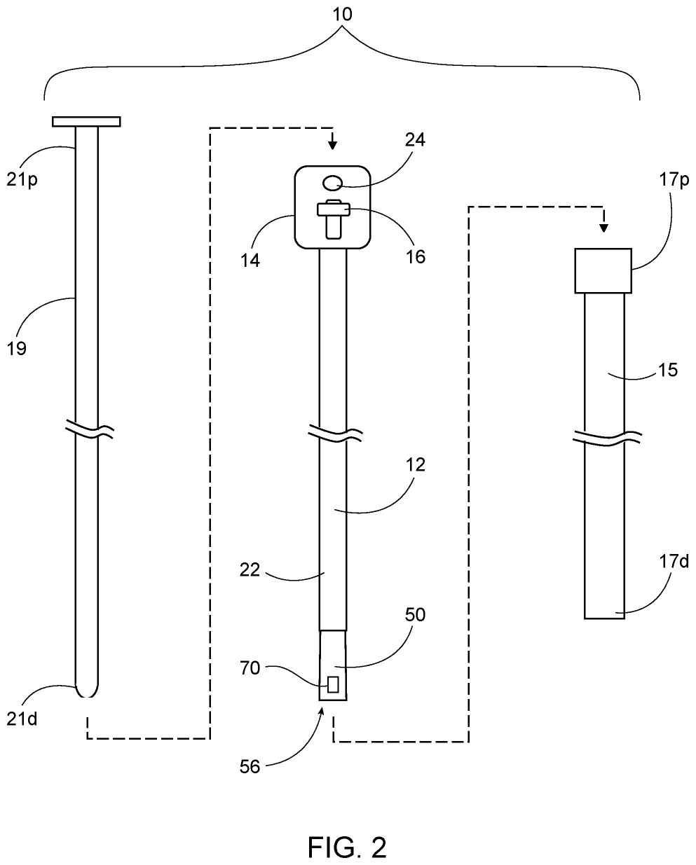

As shown in FIGS. 1 and 2, a surgical system 10 may include an hemoshield device that is configured to manage proper blood flow. More specifically, the surgical system 10 includes an expandable shield 50 that can function as a temporary one-way valve and/or filter to facilitate proper blood flow while also capturing debris as needed. The expandable shield 50 may include one or more movable and/or actuatable panels (or other structures) that are responsive to fluid flow or fluid impinging the panels in order to manage blood flow in the aorta. The expandable shield 50 may also be used to extract and capture emboli, such as water vapor, char, smoke, oxygen, nitrogen, carbon dioxide, solids, tissue fragments, etc. In one example, the expandable shield 50 can be positioned to appose the aortic wall in a manner that captures particles from the forward flow ejection of the left ventricle (LV). More specifically, the expandable shield 50 is further configured to enable adequate forward flow with the LV ejection of blood flow, by opening and closing in response to blood flow. In particular, the expandable shield 50 and its one-way valve type configuration are designed to allow diastolic flow in the direction of the coronary ostium.

While embodiments are described as a one-way valve, the surgical system may be used in combination with additional devices that are configured to a guide, capture, cut, and remove a portion of the leaflet of the valve, as described in U.S. Provisional Patent Application Ser. No. 63/324,413, filed Mar. 28, 2022, and U.S. application Ser. No. 18/127,428 filed Mar. 28, 2023, the entire contents of which are incorporated by reference to into the present disclosure. The surgical systems as described in the present application, however, may be used with other cardiovascular devices.

Continuing with FIGS. 1 and 2, the system 10 (and related methods) as described herein are configured to provide access to an aorta. The surgical system 10 may therefore include one or more distinct elements designed to guide the system toward and into the aorta and position the expandable shield 50 in place. More specifically, the surgical system 10 may include one or more of the following elements, either combined in a single assembly or comprising separate modular components: (a) a shaft 12 for targeting the system toward the desire tissue site; (b) an expandable shield 50; and in some embodiments (c) one or more actuatable panels 70 that are responsive to fluid flow. The surgical system may include a handle 14 and may include one or more actuators 16, 24 that are configured to control operation of and relative movement of elements ((a) through (c) above) of the system 10 in use.

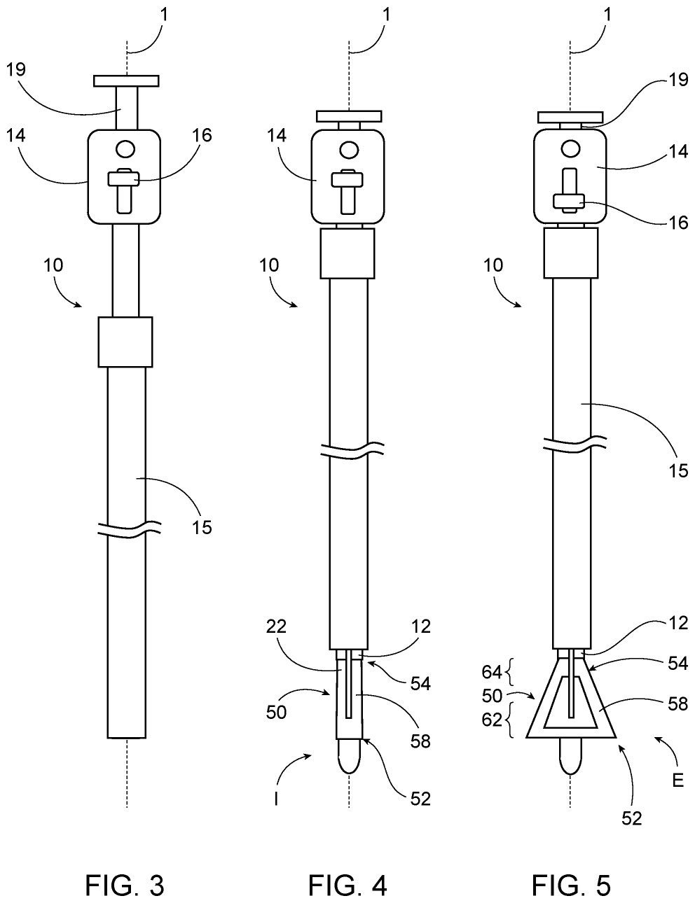

As illustrated in FIGS. 1 through 3, the surgical system 10 includes a distal end 18 and a proximal end 20 that are spaced apart from each other along a central axis 1. The system 10 may include an elongate shaft 12 that is elongated along the central axis 1. Generally, a direction from the proximal end 20 toward the distal end 18 is referred to herein as the distal direction. A direction from the distal end 18 toward the proximal end 20 is referred to herein as the proximal direction (opposite the distal direction). The surgical system 10 is configured to enter the cardiovascular system. As such an effective length of the surgical system 10, such as the portion that extends from the entry site of patient to the target location in the aorta may vary. In some examples, the effective length extends from the distal end 18 to the proximal end 20 and may range between about 40 cm up to about 120 cm, and any intervals therebetween. For instance, the effective length may between 40 cm and 80 cm, between 50 cm and 90 cm, between 60 cm and 100 cm, and any other interval between 40 cm and 120 cm. In other examples, the effective length may be larger than 120 cm. Accordingly, the surgical system size and configuration could vary as needed.

The surgical system 10, and specifically the one or more elements described above ((a) through (c) and further described below) include elongate shafts 12 that engage or are coupled to the handle 14 and are designed to extend into the aortic arch, either alone, or through a procedural sheath 15, which is typically placed in the ascending arch of the aorta to provide access to an implanted valve in the aorta. In some instances, the shaft 12 is configured as a catheter, which includes an internal channel through which other devices and elements or may pass. Its form as a catheter is not strictly required but would be useful, as needed, when coupled with other surgical devices for access to and engagement with an implanted valve in the aorta. In any event, the shaft 12 may have a trailing end 13p and a leading end 13d opposite the trailing end 13p along axis 1. The shaft 12 may have a range of outer cross-sectional dimensions, for instance, selected to fit inside a sheath 15 (described below). In particular, the distal end and shaft 12 of the system 10 may be sized to fit within the sheath 15. For example, the surgical system shaft 12 may have an outer diameter, measured perpendicular to a central axis 1 that is generally less than an internal dimension of a lumen of the sheath 15.

The surgical system 10 is generally sized and configured for insertion into a sheath positioned in the aorta. The system 10 may include additional devices, such as guide wires, introducers, etc., to facilitate introduction of the surgical system into the aortic arch. Accordingly, as shown in FIGS. 1-5, the surgical system 10 may include a sheath 15 having a distal end 17d and a proximal end 17p opposite the distal end 17d, and a lumen (not numbered) that extends from the distal end 17d to the proximal end 17b. The internal dimension or size of the access channel is sufficient to receive therein the expandable shield 50 and elongated shaft 12. The outer diameter of the sheath 15 may have an outer diameter, measured perpendicular to a central axis 1 thereof, up to about 14 F. In one embodiment, the sheath 15 may be a TAVR sheath.

The surgical system 10 may also include an introducer 19. The introducer 19 has a proximal end 21p and a distal end 21d opposite the proximal end 21p. The distal end 21d may include a tapered tip as needed. The introducer 19 may include an internal lumen sized to fit around a guide wire. In one example, the inner diameter of the introducer 19 (if used) is sized to fit around a guidewire that may be 0.035 inches. However, the inner diameter of the introducer 19 or other components, which could receive a guide wire, may vary.

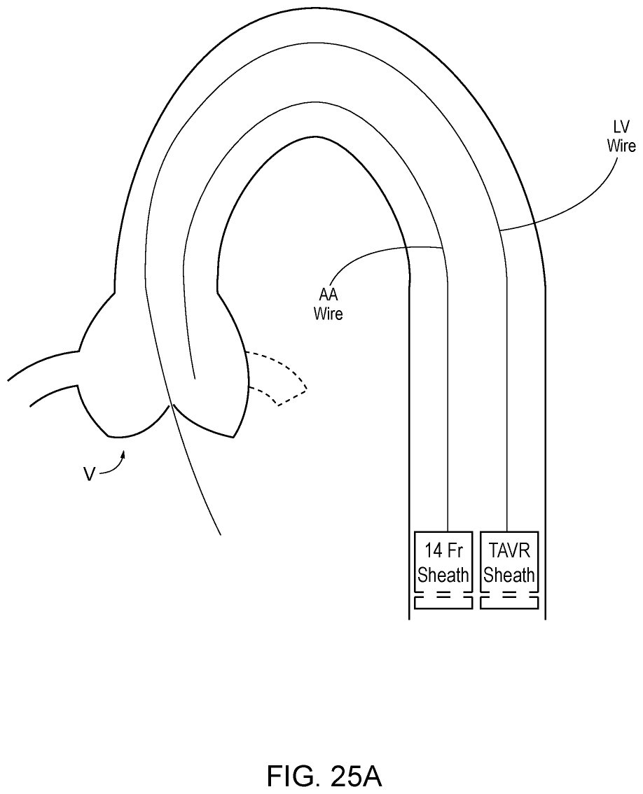

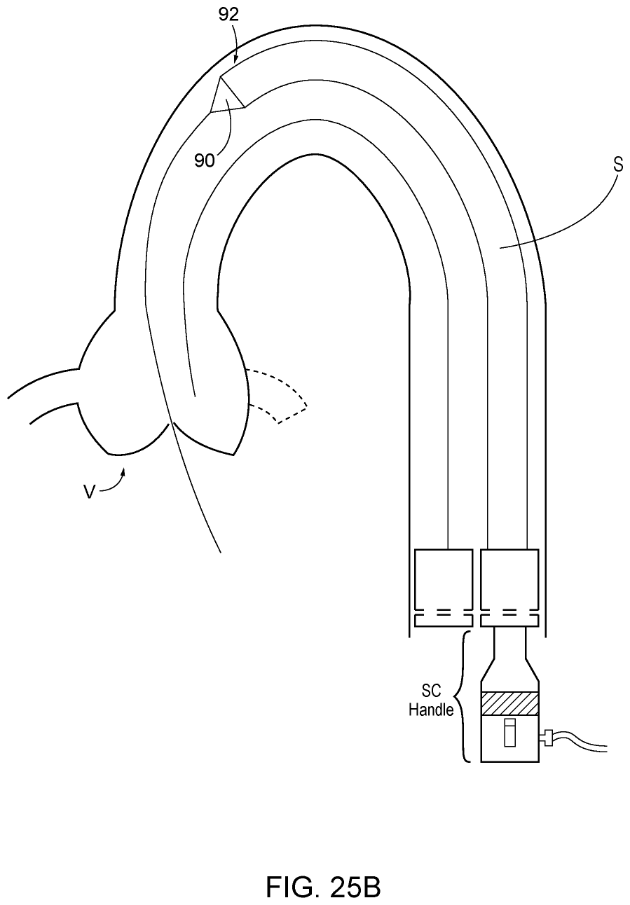

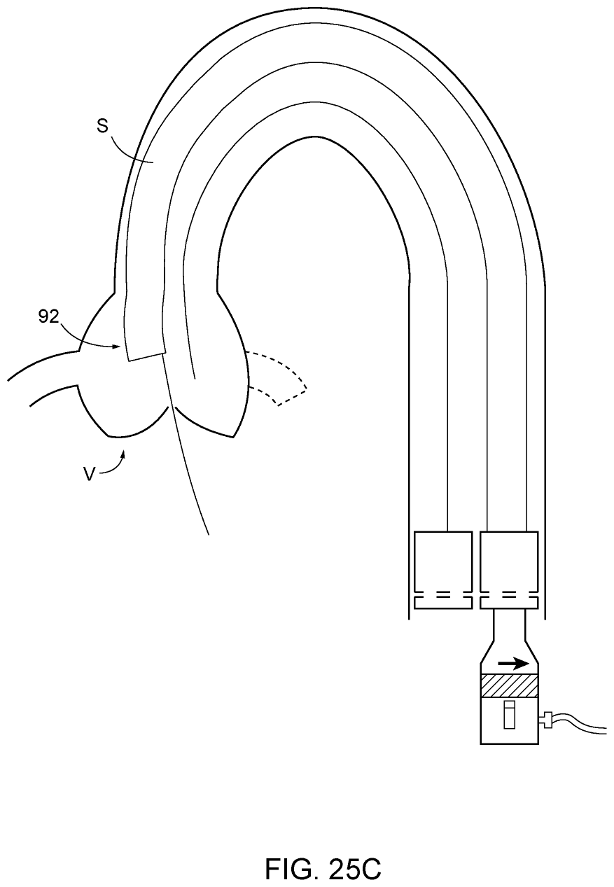

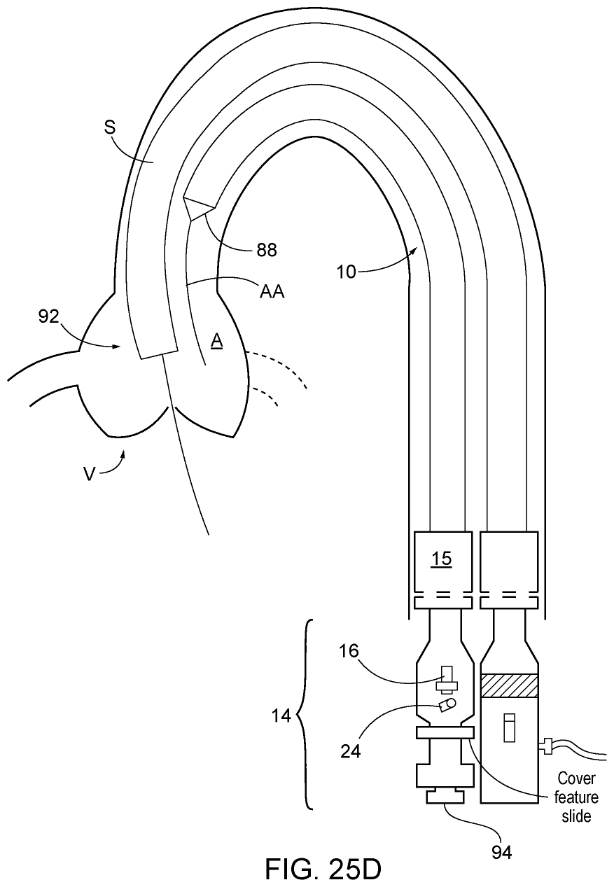

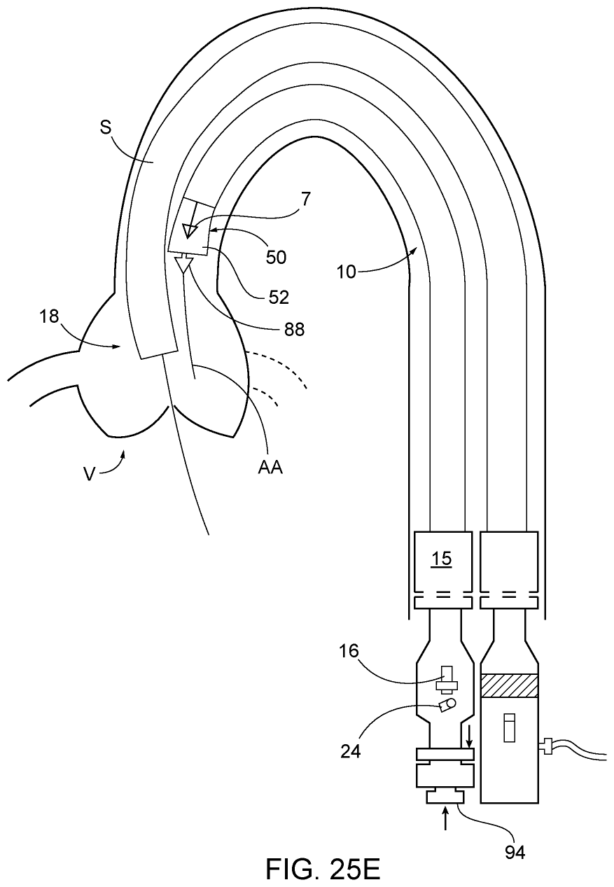

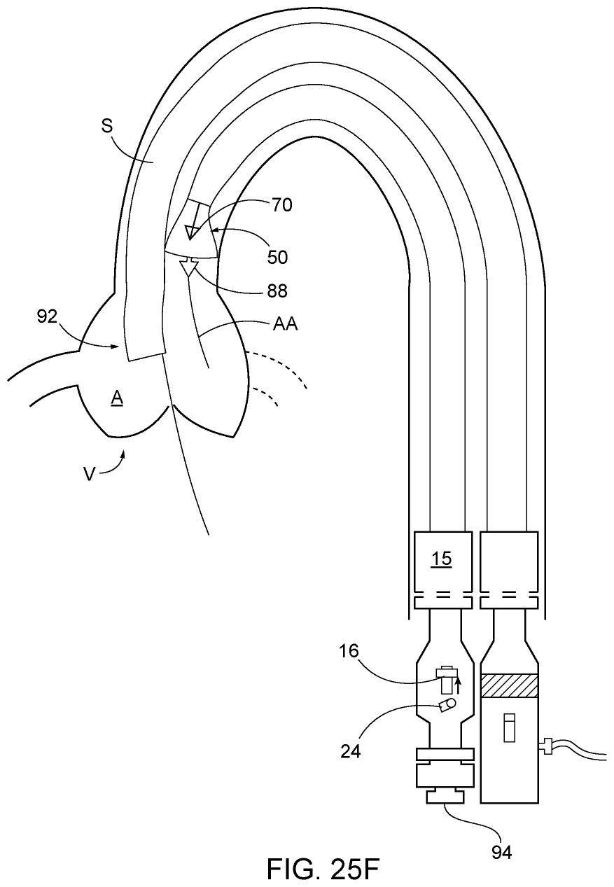

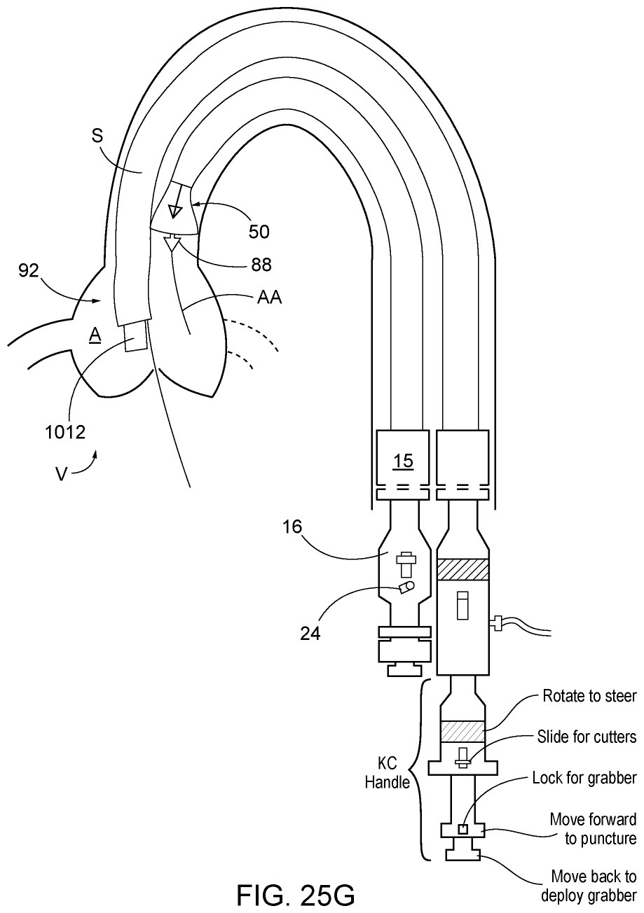

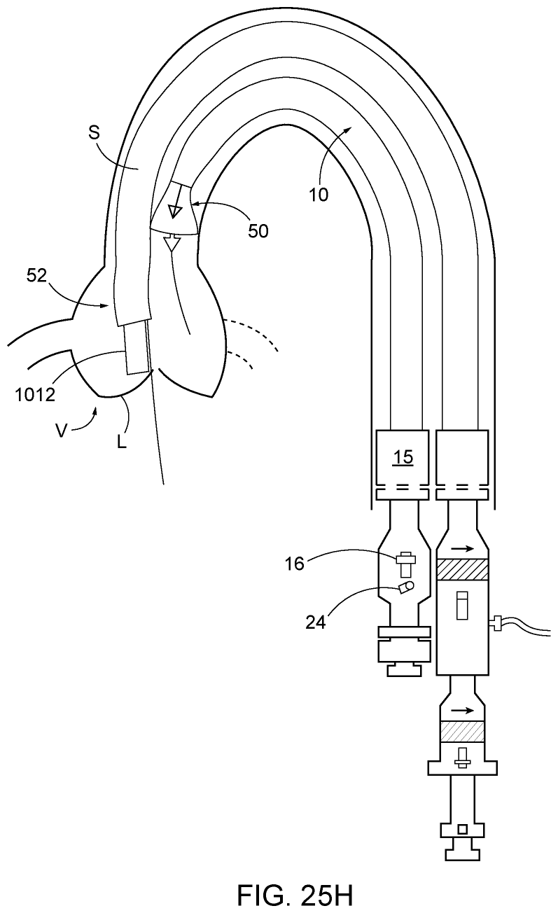

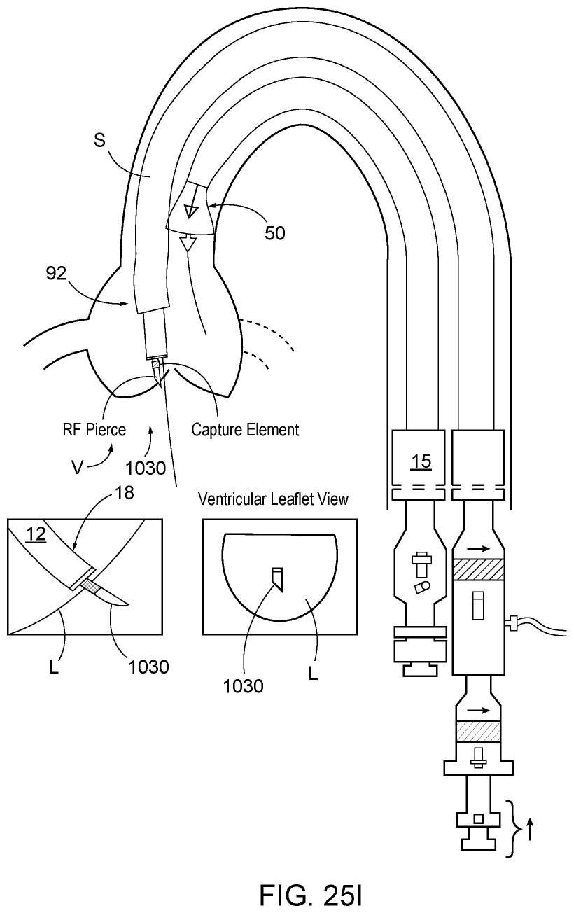

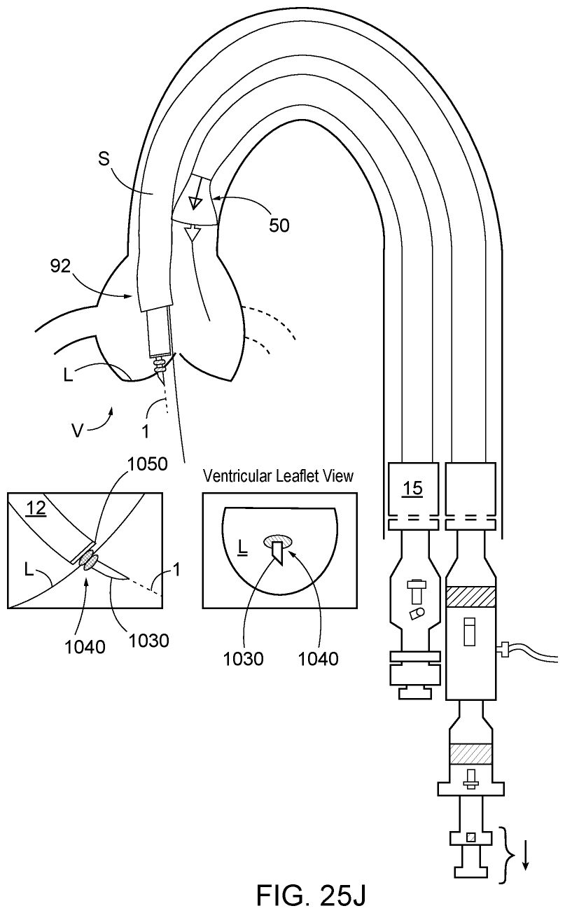

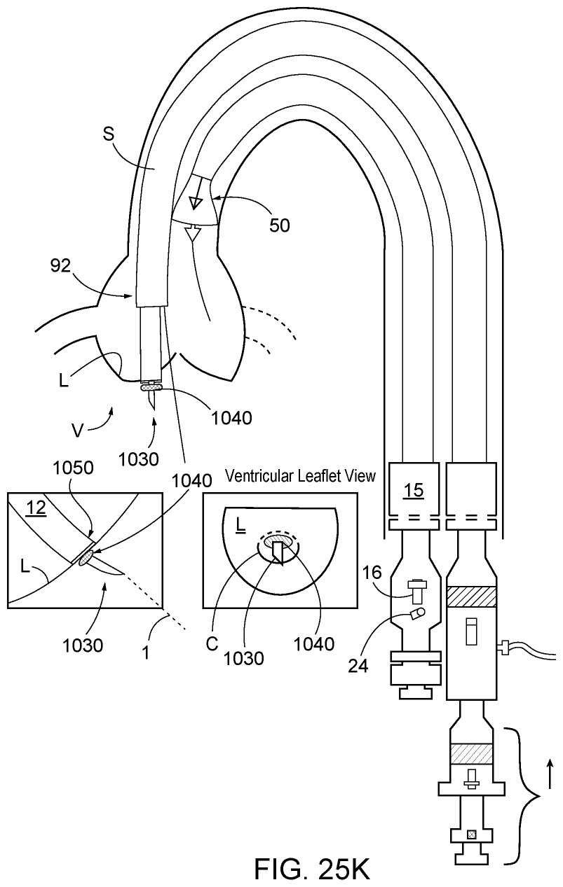

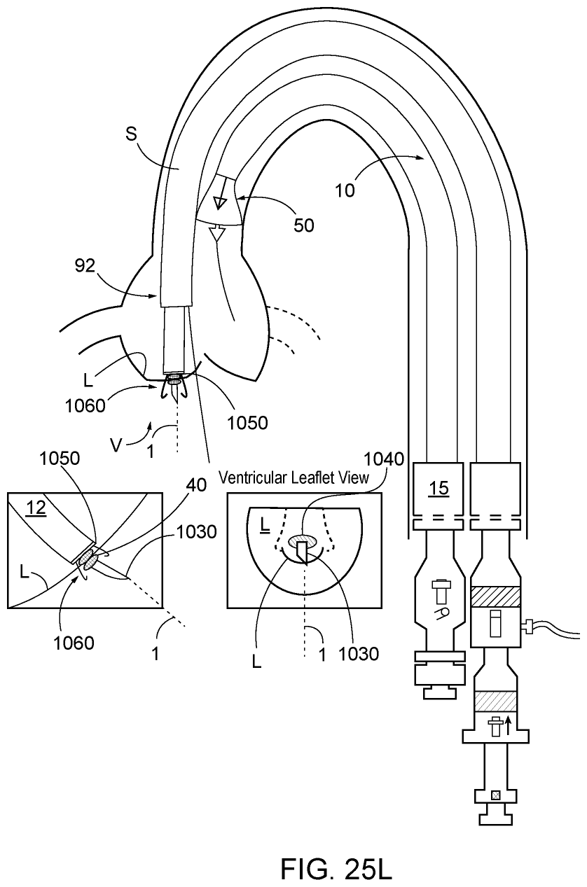

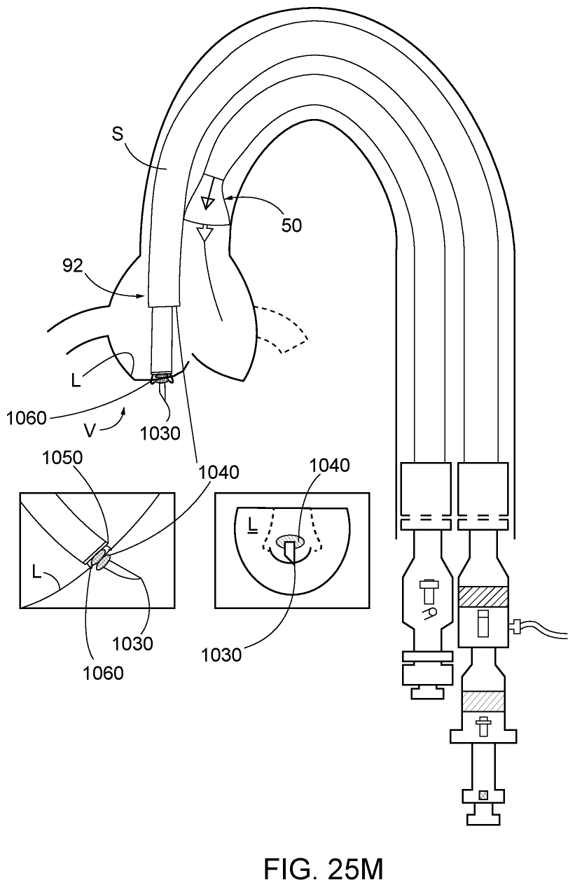

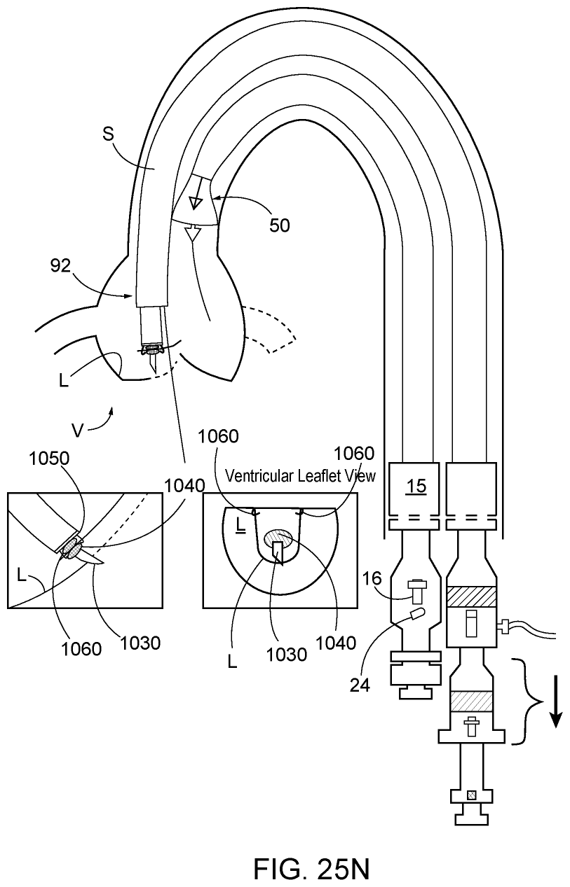

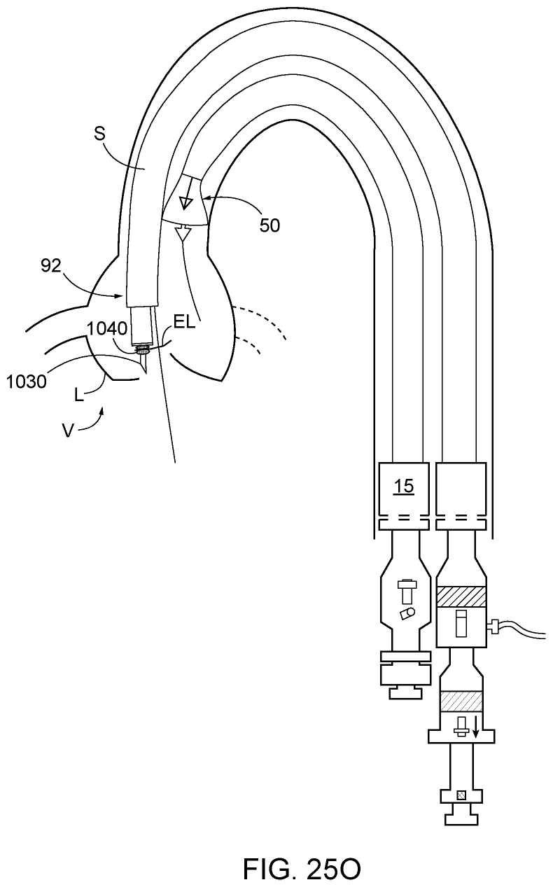

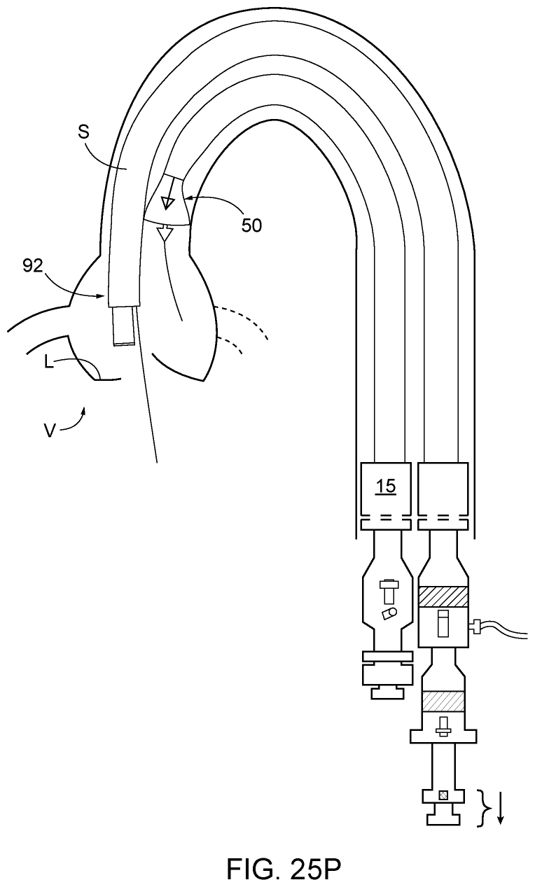

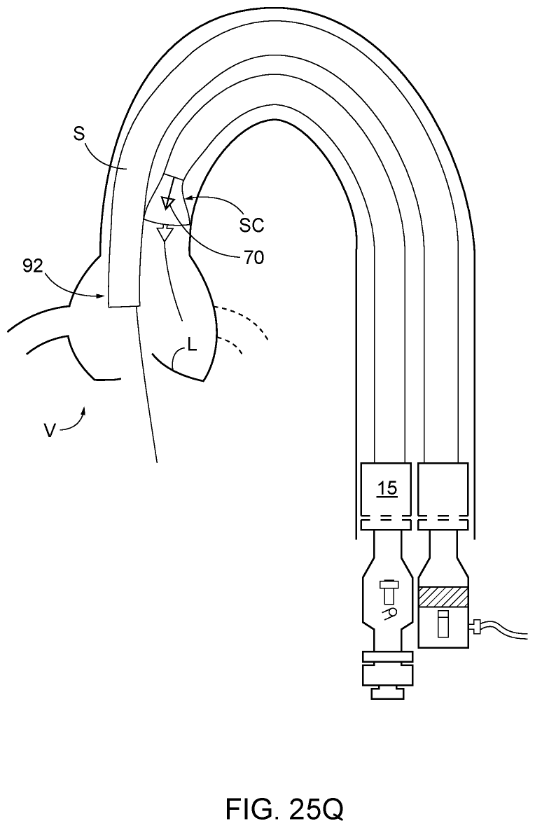

The surgical system 10 may include a nose cone 88 (FIG. 25D) carried by the elongated shaft 12 or another shaft as needed. The nose cone 88 has an outer cross-sectional dimension that is greater than an inner cross-sectional dimension of a central portion of the expandable shield 55. The nose cone 88 may include a forward tapered end. The surgical system 10 may further include a nose cone actuator 94 configured to cause movement of the nose cone 88 with respect to a leading end 52 of the expandable shield 50. In one example, the nose cone 88 is movable relative to the expandable shield 50 in at least a distal direction.

As illustrated in FIGS. 4 and 5, the surgical system 10 may include an expandable shield 50 carried by the shaft 12. The expandable shield 50 has a leading end 52 defining a distal tip 56 and a trailing end 54 that is proximal relative to the leading end 52. The expandable shield 50 is operably coupled to an actuator 16, which controls deployment of the expandable shield 50. More specifically, the expandable shield 50 is movable between an insertion configuration I, where the expandable shield 50 is collapsed toward the central axis 1, and an expanded configuration E, where the trailing end 56 expands outwardly from the central axis 1. While the expandable shield 50 in some cases may be self-expandable, actuation of the actuator 16 causes the expansion of the shield 50. In one example, the actuator 16 is a slide, advancement of which in a distal direction causes the expandable shield to expand.

The expandable shield 50 is configured to allow expansion and contraction as needed during use. As shown in FIGS. 4-7, the expandable shield 50 has a frame 58 that is configured to expand outwardly away from the central axis 1. The frame 58 defines a funnel shape in the expanded configuration E.

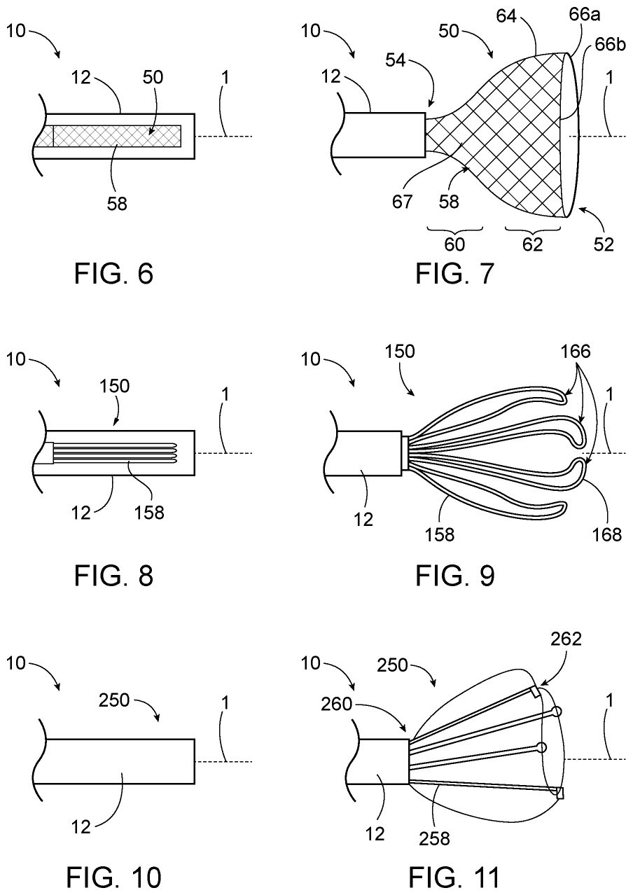

The expandable shield 50 includes a membrane coupled to the frame 58. The membrane includes actuatable and/or movable panels, as further described below, which are responsive to fluid flow. In FIGS. 4-5 the membrane outer covering is illustrated and features of the frame 58 are hidden from view while the structure of the frame 58 is best shown in FIGS. 6 and 7 without the outer covering or membrane attached to the frame 58. In any event, the membrane may include porous sections and non-porous sections. More specifically, the membrane may include a non-porous outer covering and a porous inner liner (which acts as a filter). The actuatable/movable panels are part of the non-porous outer covering, which when opened, allow fluid flow through the porous inner layer.

Referring to FIGS. 1-4, the surgical system 10 can be guided in position as needed with a steering element 22. In another embodiment, the surgical system 10 may be used in conjunction with a separate steerable catheter. The steering element 22 may be designed to be inserted through the sheath 15 and form part of the shaft 12 and includes an actuator (not shown or numbered). The steering element 22 may target the distal end 18 of the system 10 toward the desired cardiovascular structure. More specifically, as shown and described further below, the steering element 22 is configured to present the distal end 18 of the system 10 into the ascending aorta proximate the valve V so that the expandable shield 50 and actuatable panel 70 can be actuated as needed to accomplish their respective functional objectives. In such an example, the distal end of the steering element 22, for example the distal end 18 of shaft 12, can be steered or guided into position as needed to present the elements proximate the valve. In an alternative embodiment, the steering element 22, when in the form of a catheter, may also include an inner channel (not numbered) that extends between the distal and proximal ends of shaft 12. A catheter is not strictly required to effectuate steering or targeting as described herein. While a sheath 15 and steering element 22 are described as separate components, the surgical system may combine these two features into a single device whereby the sheath is steerable.

As illustrated in FIGS. 6 and 7, the frame 58 has a proximal portion 60 coupled to the shaft 12, a distal portion 62 opposite and distal to the proximal portion 60, and at least one support bar 64 that couples the proximal portion 60 to the distal portion 62. The distal portion 62 expands outwardly away from the central axis 1 while the proximal portion 60 does not expand outwardly. The frame 58 includes a first wire 66a defining a first portion of a leading end 52 of the expandable shield 50 and a second wire 66b defining a second portion of the leading end 52 of the expandable shield 50. The frame 58 further includes a plurality of support wires 67 coupled to the first wire 66a and the second wire 66b. The first wire 66a and second wire 66b expand radially outwardly into a ring shape. While two wires 66a, 66b are shown forming the leading end 52 when expanded, the leading end 52 can be formed from more than two wires, that, when in the expanded shape can form a generally ring shape. Adjacent wire ends may overlap as need to better define the ring shape.

FIGS. 8-11 illustrate alternative embodiments of expandable shields 50. Thus, while expandable shields shown in FIGS. 8-11 differ from the shield and frame structure shown in FIGS. 6 and 7, there are common features for each shield and like reference numbers as used to identify those common features and structure. As illustrated in FIGS. 8 and 9, an expandable shield 150 includes the frame 158 having a plurality of separate flexible arms 166. Each arm 166 defines a terminal end 168 such that the terminal ends 168 of the separate flexible arms 166 expand outwardly away from the central axis 1 during expansion. As illustrated in FIGS. 10 and 11, the shield 250 includes a frame 258 that is a self-expanding wire mesh. Here, the frame 258 has a distal portion 262 that expands radially outwardly and a proximal portion 260 at a distal end of the shaft 12 that remains collapsed toward the central axis 1. The wire mesh includes a terminal outer ring (not numbered) and multiple wires extending from the outer ring to the distal end of the shaft 12.

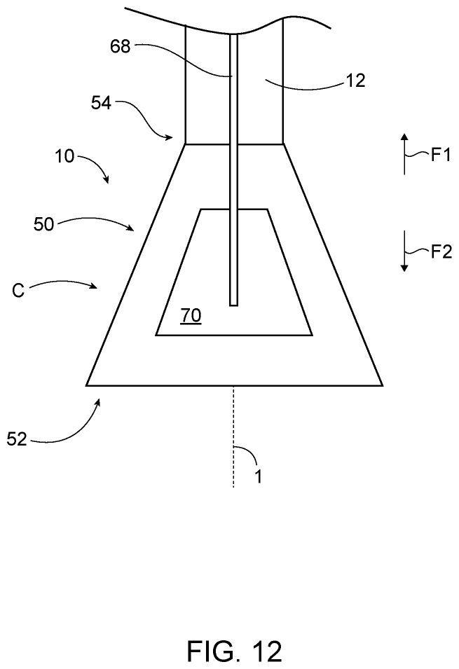

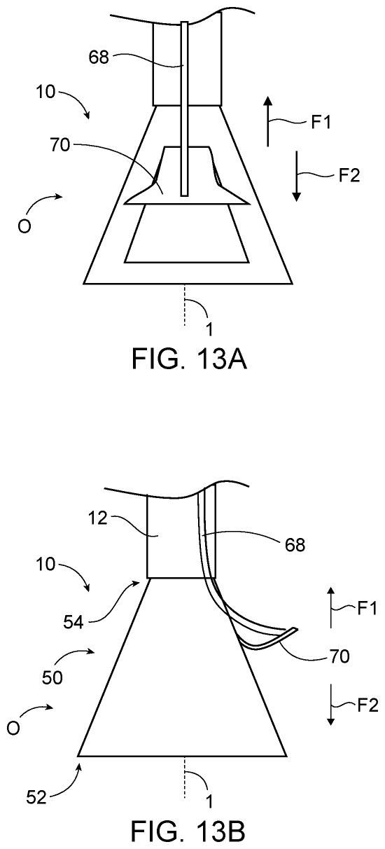

Regardless of the specific construction of the frame 58, 158, 258, as shown in FIGS. 12-14, the surgical system 10 includes an actuatable panel 70 coupled to the expandable shield 50. The actuatable panel 70 is configured to transition between A) a closed configuration C (FIG. 12) that inhibits fluid flow through the expandable shield 50 in both a first direction F1 and a second direction F2 that is opposite the first direction, and B) an open configuration O (FIGS. 13A and 13B) that permits fluid flow through the expandable shield 50 in both the first direction F1 and the second direction F2. One or more actuators may be disposed on the handle 14 (FIG. 1) to control transition of the actuatable panel 70 between and among the closed configuration C and the open configuration O. More specifically, at least one actuator 24 coupled to the actuatable panel 70 is configured to control operation of the actuatable panel 70 to permit or inhibit fluid flow through the expandable shield 50. In this manner, the actuator 24 can be used to control regurgitation.

Referring to FIGS. 12-14, the surgical system 10 may include an actuation rod 68 that may be coupled to the actuatable panel 70 and the actuator 24. This construction is selected so that actuation of the actuator 24 causes the actuation rod 68 to move the actuatable panel 70 between the closed configuration C and the open configuration O. In an embodiment, an alignment element (not shown or numbered) may be coupled to the actuation rod 68 and the actuatable panel 70. The alignment element may help guide the actuation and better control movement of the panel 70.

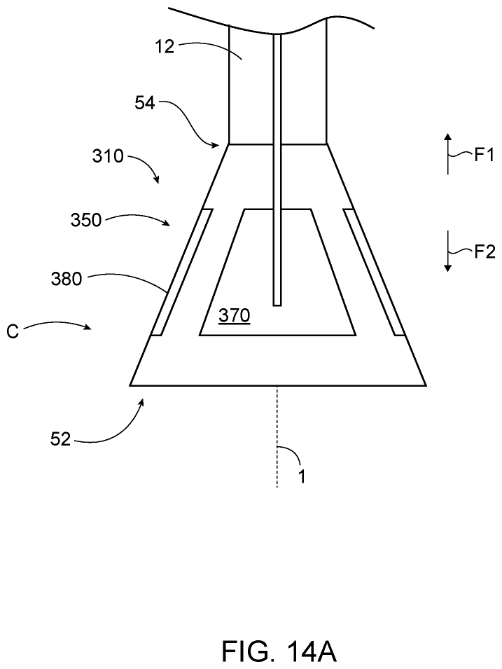

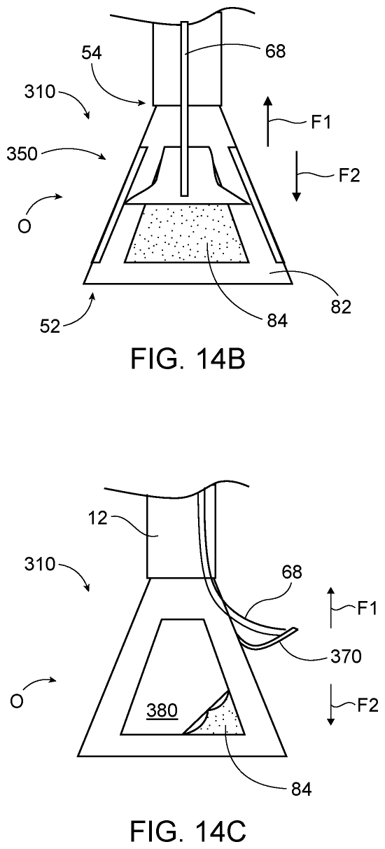

In accordance with another embodiment of the present disclosure, the surgical system 310 may include an alternative expandable shield 350 with both an actuatable panel 370 and a movable panel 380. The surgical system 10 shown in FIGS. 1-13B is similar to the surgical system 310 shown in FIGS. 14A-14C and common reference numbers are used to identify features that common between surgical system 10 and surgical system 310. As such, the surgical system 310 includes the shaft 12 elongated along a central axis 1, an expandable shield 350 carried by the shaft, a movable panel 380 on the expandable shield 350, and an actuatable panel 370 that overlies the expandable shield 50. The expandable shield 350 has a non-porous section 82 and a porous section 84 (or multiple porous sections) with the movable panel 380 overlying the porous section 84 in the closed configuration.

As described above, the expandable shield 350 has a leading end 52 a trailing end 54 that is proximal relative to the leading end 52. In this embodiment, the expandable shield 350 is movable in response to fluid flow. More specifically, the expandable shield is movable between an insertion configuration (similar to that shown FIG. 2), where the expandable shield 350 is collapsed toward the central axis 1 in response to a first direction of fluid flow, and an expanded configuration, where the trailing end is expanded outwardly away from the central axis 1 in response to a second direction of fluid flow. While the expandable shield 350 described above is configured so that expansion occurs in response to fluid flow, the expandable shield 350 can be configured to self-expanded, similar to the expandable shield 50 described above.

Referring to FIGS. 14A-14C, the movable panel 380 is configured to open in response to fluid flow in a first direction that impinges the expandable shield 350, and close in response to fluid flow in a second direction that is opposite the first direction that impinges the expandable shield 350. The movable panel 380 is therefore movable between an open configuration O (FIG. 14C partially open) and a closed configuration C (FIG. 14A). In one embodiment, the movable panel 380 is configured to rotate into the open configuration. In another embodiment, the movable panel 380 is configured to slide into the open configuration.

The expandable shield 350 may include a plurality of movable panels 380 that overlie a plurality of porous sections 84, respectively. In this example, each movable panel 380 is independently responsive to fluid flow to open or close. In the illustrated embodiment, the movable panel 380 is configured such that 1) fluid flow in the first direction opens the movable panel 380 to permit fluid to flow through the porous section, and 2) fluid flow in the second direction that is opposite the first direction causes the movable panel 380 to close to inhibit fluid flow through the porous section 84. That is, the panel 380 opens or closes in response to fluid flow while the actuatable panel 370 can controlled by the user to open or close as needed.

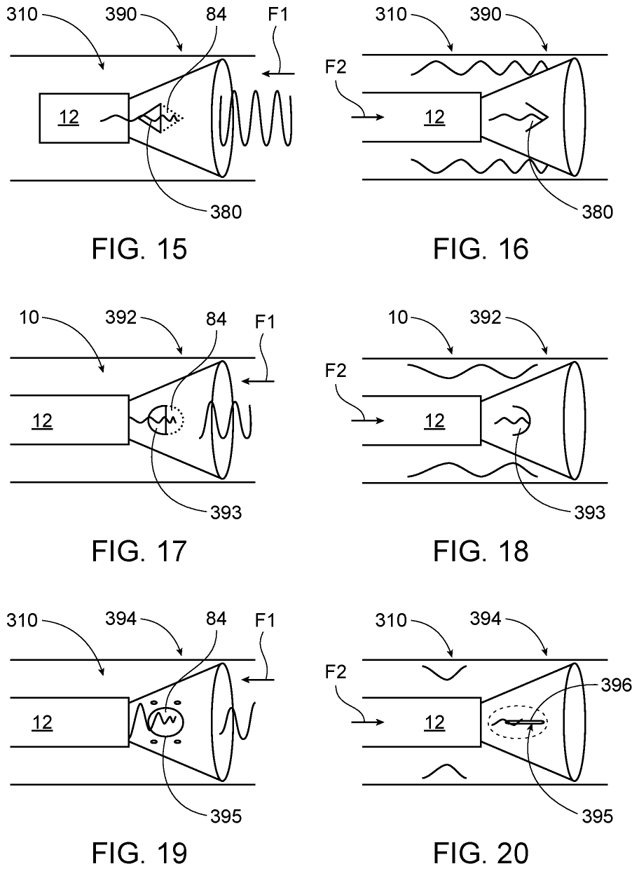

Furthermore, the surgical system 310 may include moveable panels with a variety of different configurations. The expandable shields shown in FIGS. 15-20 are similar to the expandable shields 50 and 350 and therefor common references numbers are used to identify features common to expandable shields shown in FIGS. 14A-14C and FIGS. 15-20. Each shield shown in FIGS. 15-20 is carried by the distal end of the shaft 12. As illustrated in FIGS. 15 and 16, the expandable shield 390 may have a movable panel 380 (which may be a tapered panel) that overlies a porous section 84 of the expandable shield 390. Fluid flow in the first direction F1 opens the tapered movable panel 391 to permit fluid to flow through the porous section 84 (FIG. 15). Fluid flow in the second direction F2 that is opposite the first direction causes the tapered movable panel 391 to close to inhibit fluid flow through the porous section 84 (FIG. 16).

As illustrated in FIGS. 17 and 18, the expandable shield 392 may include a movable panel 392 that is a curved panel 393 that overlies a porous section 84 of the expandable shield 392. Fluid flow in the first direction F1 opens the curved movable panel 393 to permit fluid to flow through the porous section 84 (FIG. 17). Fluid flow in the second direction F2 that is opposite the first direction causes the curved movable panel 393 to close to inhibit fluid flow through the porous section 84 (FIG. 18).

As illustrated in FIGS. 19 and 20, the expandable shield 394 may include movable panel 395 includes at least one slit 396 that overlies a porous section 84 of the expandable shield 394. Fluid flow in the first direction F1 opens the at least one slit 396 to permit fluid to flow through the porous section 84 (FIG. 19). Fluid flow in the second direction F2 that is opposite the first direction causes the at least one slit 396 to close to inhibit fluid flow through the porous section 84 (FIG. 20).

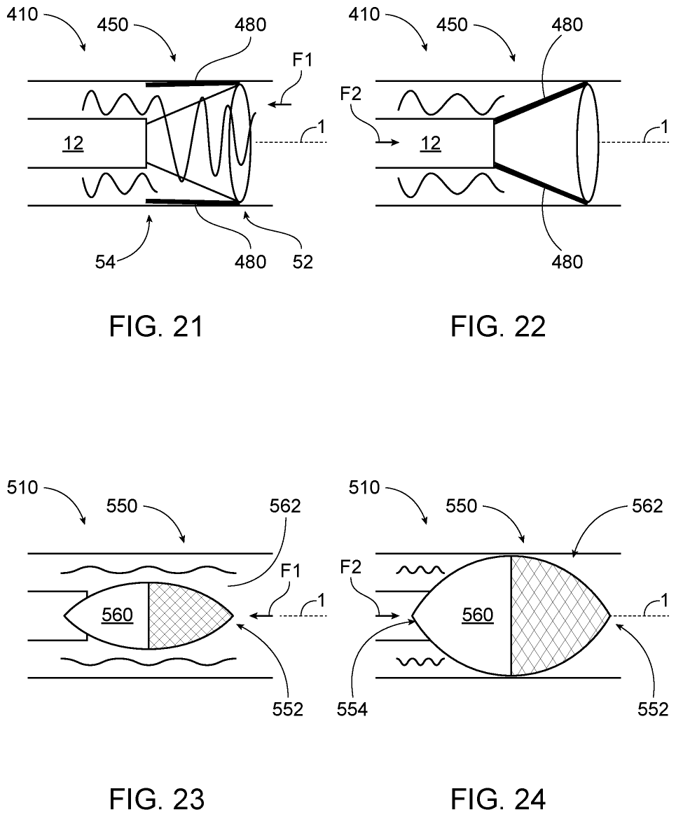

FIGS. 21 and 22 illustrate yet another embodiment of a surgical system 310 according to an embodiment of the present disclosure. The surgical system 410 may include an expandable shield 450. Features common to surgical system 10, 310 and 410 will use common reference numbers to identify features common to all the described embodiments. In addition, the expandable shield 450 shown in FIGS. 21 and 22 may be similar to the expandable shields shield 50, 350, 390, 392, 394 described above and therefor common references numbers are used to identify features common to expandable shields shown in FIGS. 14A-20 and FIGS. 21 and 22. As illustrated in FIGS. 21 and 22, the expandable shield 450 is carried by an elongated shaft 12. The expandable shield 450 includes one or more movable panels 480 pivotably coupled to leading end 52 of the shield 450. The moveable panels 480 in this example are coupled to the leading end 52 so that they can pivot outwardly in response to fluid flow. As shown in FIG. 21, fluid flow in the first direction F1 opens the movable panels 480. In FIGS. 22, fluid flow or in the second direction F2 (opposite the first) closes the fluid panels 480.

As illustrated in FIGS. 23 and 24, a surgical system 510 is shown having an expandable shield 550 has a leading end 552 defining a tapered tip 556 and a trailing end 554 that is proximal relative to the leading end 552. The expandable shield 550 is configured to transition between an insertion configuration, where the expandable shield 550 is collapsed toward the central axis 1 in response to a first direction F1 of fluid flow. The trailing end 552 expands outwardly in response a second direction F2 of fluid flow. In this embodiment, the expandable shield 550 functions as a parachute that opens to inhibit flow and closes to permit fluid flow. In this case, proximal portion 560 is non-porous section and a distal portion 562 may be a porous section, each coupled to frame (not numbered). To open, the fluid flow impinges the proximal portion, causing that portion to expand outwardly.

Each of the surgical systems as described herein may include an elongated shaft 12. In some examples, one or more of the elongate shafts described herein could be in the form of a catheter. For example, an outer shaft as shown in FIGS. 1 and 2 may be a catheter with an inner channel that includes features to carry and guide elongate shafts of the expandable shield.

The shafts described herein, when in the form of catheters will generally include a shaft, an inner channel, one or more radiopaque markers, and a distal tip. One of or more catheters as described herein may have a secondary curve, a primary curve, or no pre-set curves. The primary and secondary curves are not illustrated in the drawings. The distal tip defines the distal most end of each elongate shaft. The shaft may, for example, include an inner channel that is also sized to receive other surgical devices therethrough. For example, the surgical system 10 can receive a guidewire such that an over-the-wire technique may be used. That is, a guidewire can be placed through the valve structure into the left ventricle and the distal end of the surgical system 10 inserted over the guidewire into position. In an alternative embodiment, the surgical system 10, or one or more of its shafts, may include one or more skive ports that can be used to receive the guidewire therethrough. Such skive ports may be disposed toward or along an outer surface of the shaft. In yet another embodiment, the guidewire may not extend through the valve structure into the ventricle. The surgical system, however, may still slide over or along the guidewire, but without the benefit of having the guidewire cross through the valve structure.

In cross-section, a catheter may include an inner liner, a middle reinforcing layer (e.g. a braid), and an outer layer or outer jacket. In addition, the catheter may be a biaxial design that includes an additional outer layer to minimize interaction with the introducer and/or sheath and allow smoother movement of the surgical system. In another embodiment, the catheter would also be able to accommodate different shaped inner catheters to achieve a suitable relationship of the distal catheter tip to the leaflet. For example, this configuration may provide for functionality similar to the use of a 5 F/6 F 120 mm IM catheter inside an AL type catheter, i.e. a mother and daughter technique. The catheter may be configured to transition in response to operator input to assume different degrees of flexion of the distal tip to account for different patient anatomy.

The longitudinal shape of the catheter can vary as needed. For instance, the catheter can have a shape according to the Amplatz Guide that includes, but is not limited to AL-1, AL-2, AL-3, AL-4, etc. Other common shapes are possible as well. In one example, the catheter may have an outer cross-sectional dimension sized for insertion into the aorta. For instance, the catheter may be either 12 French or 14 French. However, larger or smaller sized catheters may be used in certain instances. The catheter tip or distal tip may be deflectable or bendable as needed to steer the distal tip into position, for example, when using a steering element as described above. The catheter may also be configured to accommodate different shaped inner catheters.

The catheter has at least one port that extends to the inner channel. As shown, the at least one port could be two or more as needed. The port or ports are spaced a distance from the leading end that is less than a distance between the at least one port and the trailing end. In other words, they are positioned toward the leading end of the catheter. These ports are intended to a) allow for flushing or priming the system prior to introduction to the patient and/or b) allow removal of emboli, such as air and other debris after cutting, and throughout, to provide for hemodynamic monitoring of the blood pressure in the ascending aorta. For instance, when the leaflets get cut, the destruction of the aortic valve may lead to decompensation of coronary output, which is monitored by a local lumen. The system may, in turn, include a luer fitting on the handle for monitoring and bubble removal. Bubble and debris removal can happen via an active ‘vac-lok’ syringe (pull a vacuum with a syringe and the handle locks in place so holding by the user is not required) on the port for evacuating 50-100 ml of blood/air.

The present disclosure includes various embodiments for controlling flow in a cardiovascular system while performing a surgical procedure. One example of such a procedure includes extracting a portion of a leaflet of a valve, e.g. a surgical valve or TAVR, as described in U.S. Provisional Patent Application Ser. No. 63/324,413, filed Mar. 28, 2022, and U.S. application Ser. No. 18/127,428 filed Mar. 28, 2023, mentioned above. To that end, FIGS. 25A-25Q illustrate an exemplary method for the controlling flow during surgical procedure while excising a leaflet of a valve. While a particular surgical procedure is shown used with the surgical system 10 in FIGS. 25A-25Q, the surgical system 10 may be used with other procedures. Thus, the hemoshield and related surgical systems as described herein are not limited strictly for use in a surgical procedure that includes excising a leaflet portion of a valve.

Referring to FIG. 25A, the method may generally include placing one or more sheaths in the cardiovascular system, e.g. the ascending aorta. The sheaths may include a sheath for the surgical system and sheath for the related surgical procedure. Guidewires LV and AV may be inserted into the sheaths and their distal ends positioned proximate or through the valve.

As shown FIGS. 25B-25C, the method may include advancing a steerable shaft S with a dilator 90 into a cardiovascular system so that its distal end 92 is proximate a valve. The dilator 90 is then removed. Next, as shown in FIG. 25D, the user may insert surgical system 10, including sheath 15 and introducer 19 toward the aorta A.

In FIG. 25E, the expandable shield 50 is advanced, in an insertion configuration, along the guidewire AA to a location in the ascending aorta A. In one embodiment, the nose cone 88 and expandable shield 50 may be pushed forward. Next, as shown in FIG. 25F, the user may expand the expandable shield 50 from the insertion configuration into the expanded configuration such that an outer perimeter of the expandable shield 50 is positioned outwardly toward an aortic wall. At this point in the procedure, a user may actuate the actuatable panel 70 overlying the porous section of the expandable shield 50, between an open configuration and a closed configuration, to selectively permit blood flow through the porous section of the expandable shield 50. This, in effect, is a mechanism to control regurgitation and flow. In addition, the user may actuate the actuatable panel 70 overlying a porous section of the expandable shield 50, between an open configuration and a closed configuration, to selectively permit blood flow through the porous section of the expandable shield 50. In yet another embodiment, the user may further allow the movable panel 80 (not shown in FIG. 25G) to open and close in response to systolic and diastolic blood flow.

Next, as shown in FIG. 25G, a catheter S may be advanced next to the guidewire LV. More specifically, a user may cause a shaft 1012 to advance out of the catheter S. As shown in FIG. 25H, the user may further advance the distal end of the shaft 1012 adjacent the leaflet L. In FIG. 25I, the method includes advancing a piercing element 1030 along into contact with a leaflet L of the valve V. Then, the piercing element 1030 may be advanced in a distal direction to form a pierced opening in the leaflet L of the valve. In one example, but not required, forming the pierced opening with the piercing element 1030 comprises supplying electrical energy to piercing element when in contact with the leaflet.

As shown in FIG. 25J, the user may advance a capture element 1040 along the central axis 1 and through the pierced opening in the leaflet L. Advancing the capture element 1040 through the pierced opening in the leaflet L will further include deploying the capture element 1040 through the pierced opening in an insertion configuration where a first expandable portion and a second expandable portion of the capture element are collapsed toward a central axis. Then, the user can transition the capture element 1040 from an insertion configuration into a capturing configuration, where the capture element 1040 seizes the leaflet.

As shown in FIG. 25K, with the leaflet L seized by the capture element 1040, the surgical system can form a curved cut C in the leaflet proximate the pierced opening with a first cutting element 1050. Forming the curved cut C includes advancing the first cutting element 1050 into contact with the leaflet proximate the pierced opening. In one example, forming the curve cut includes supplying electrical energy to the first cutting element 1050 when in contact with the leaflet. In these examples, the curved cut extends around a portion of the central axis. For instance, the curved cut is substantially C-shaped and/or extends around a majority of the central axis.

As shown in FIGS. 25L-25N, while seizing the leaflet with the capture element 1040 to maintain the relative position of the leaflet relative to the shaft 1012, a second cutting element 1060 is inserted through the leaflet. The second cutting element 1060 has first and second cutters, as described above. More specifically, the first cutter and the second cutter are advanced into contact with the leaflet. Then, the first cutter and the second cutter may splay apart while supplying electrical energy to the first and second cutters. During the step, the first and second cutters are also retracted to excise the leaflet, thereby forming an excised portion of the leaflet. Furthermore, during the splaying, the user retracts the capture element, while seizing the cut leaflet portion, toward the proximal end of the steerable shaft S.

While the first and second cutters are in the cutting configuration and moving in the proximal direction, the capture element 1040 maintains its relative position while still grabbing the leaflet L. By placing some distally directed force on the capture element, tension is maintained in the leaflet, allowed the first and second cutters to splay apart, either through shape memory, or by following the natural anatomy of the valve. The first and second cutters separate and splay, while moving in the proximal direction, excising a portion of the leaflet L (excised portion not shown). By holding or advancing the leaflet L in place while splaying the first and second cutters, the amount of leaflet that can be removed from the valve may be optimized. In other words, holding the leaflet while splaying the cutters to excise the leaflet can maximize the amount of tissue that can be removed from a particular leaflet. In this regard, the excised leaflet remains attached to the capturing element for subsequent removal from the patient as depicted in FIGS. 25O through 25Q.

As also shown in FIG. 25Q, the user may further allow the movable panel 70 to open and close in response to systolic and diastolic blood flow in order to adjust flow. The user may then retract and remove the excision catheter and leaflet. In addition, though not shown, the user can cause the expandable shield to collapse and retract back into the sheath 15, permitting further removal of the surgical system 10 from the cardiovascular system.

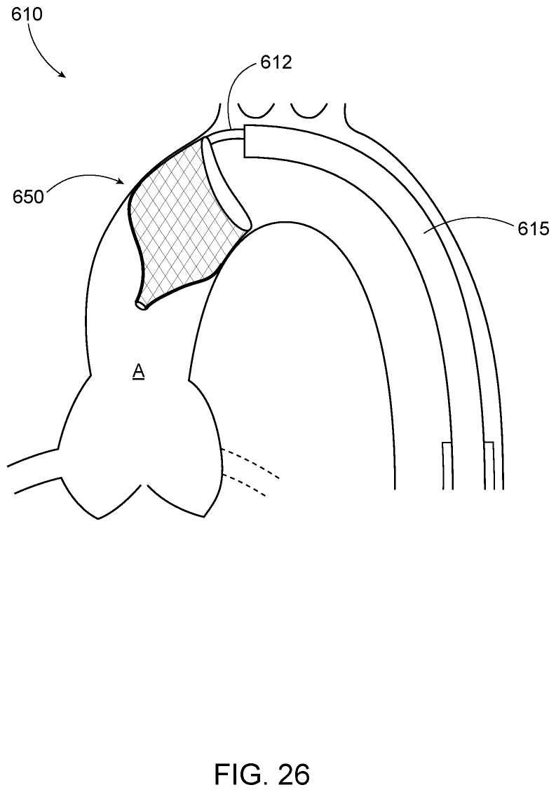

Referring to FIGS. 26-30, in another embodiment, a surgical system 610 is configured to manage fluid flow in an aorta A during a surgical procedure. The surgical system 610 may include, as with other embodiments of the present disclosure, a sheath 615 and a shaft 612. An expandable shield 650 is carried by the distal end of the shaft 612.

As shown in FIGS. 26-28, the expandable shield 610 has a leading end 652, a trailing end 654 spaced rearward of the leading end 652, an internal side 656, an external side 658 that opposes the internal side 656, and an expandable wire frame 670 that defines the trailing end 654. The trailing end 654 is expandable outwardly away from the central axis 1 to open the expandable shield 650 into the expanded configuration. The movable panel 680 is located at the leading end 652 and coupled to the internal side 656 of the expandable shield 650, such that fluid flow that impinges the movable panel 680 causes a leading end 652 of the expandable shield 650 to contract toward the central axis 1. In the contracted state, the expandable shield 650 permits fluid flow.

In the illustrated embodiment in FIGS. 26-28, the expandable shield 650 is an expandable/invertible braid and is sized to fit in at least 12 French catheters, e.g. the shaft 612b. In another embodiment, the expandable shield 650 may be sized to fit in catheters smaller than 12 French. The expandable braid may be lined with a non-porous membrane to ensure morphological changes occur appropriately to maintain forward blood flow. The expandable shield 650 may also include loops 672a, 672b at either the leading end 652 an/or trailing end 654 to capture debris. The tip 674 of the expandable shield 650 may be a nitinol shape memory alloy, which allows for necking at the leading end 652. The tip 674 may be outwardly expandable such that one or more catheters may pass through the tip 674, as needed.

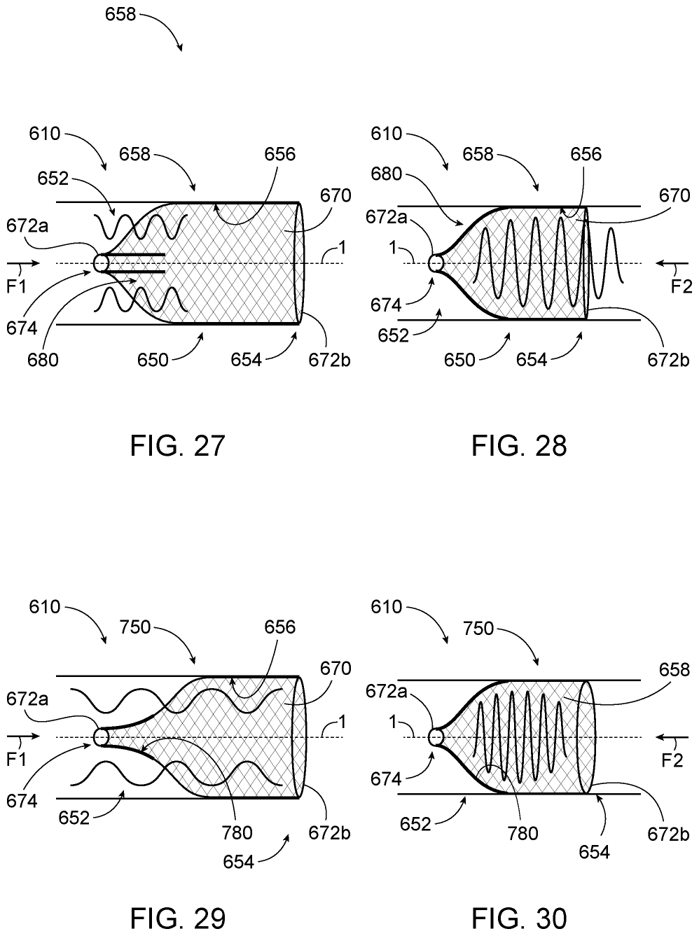

In this embodiment (FIGS. 26-28), however, fluid flow in the first direction F1 impinges the movable panel 680 causing the leading end 652 of the expandable shield 50 to contract toward the central axis 1, thereby permitting fluid flow therethrough. (FIG. 27). Fluid flow in the second direction that is opposite the first direction causes the movable panel 680 to expand outwardly and inhibits fluid flow (FIG. 28).

Referring to FIGS. 29-30, another embodiment of an expandable shield 750 is shown. The expandable shield 750 is similar the expandable shield 650 shown in FIGS. 26-28 and the same reference numbers are used to identify features that common to both shields 650 and 750. In this embodiment, however, the movable panel 780 is coupled to the external side 658 of the expandable shield 50, such that fluid flow that impinges the movable panel 780 causes a leading end 652 of the expandable shield 750 to contract toward the central axis 1, thereby permitting fluid flow. More specifically, fluid flow in the first direction F1 impinges the movable panel 380 causes a leading end 652 of the expandable shield 50 to contract toward the central axis 1, thereby permitting fluid flow therethrough. (FIG. 29). Fluid flow in the second direction F2 that is opposite the first direction causes the movable panel 780, and the expandable shield to which it is coupled, to move outward, away from the central axis 1, permitting fluid flow and inhibit fluid flow (FIG. 30).

In either embodiment 650 or 750, a guidewire may be positioned through a central portion or tip 674 of the expandable shield 650, 750 such that the expandable shield 650,750 is slidable along the guidewire.

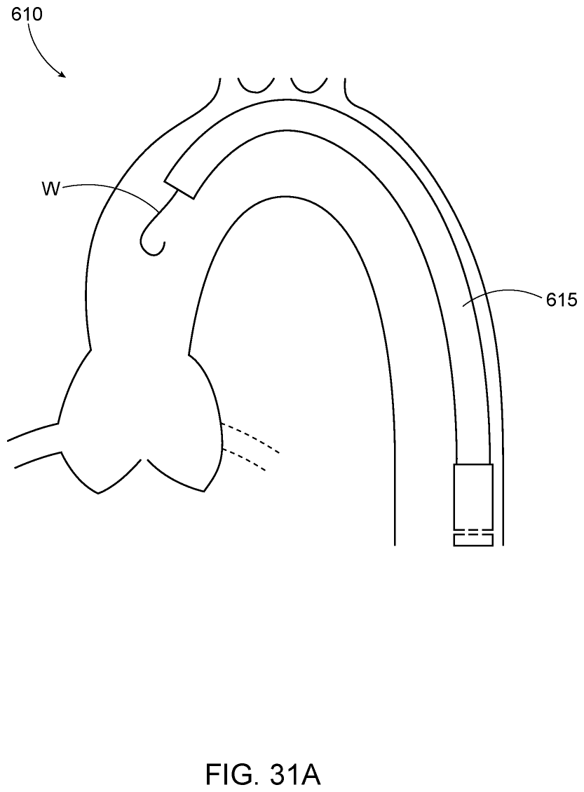

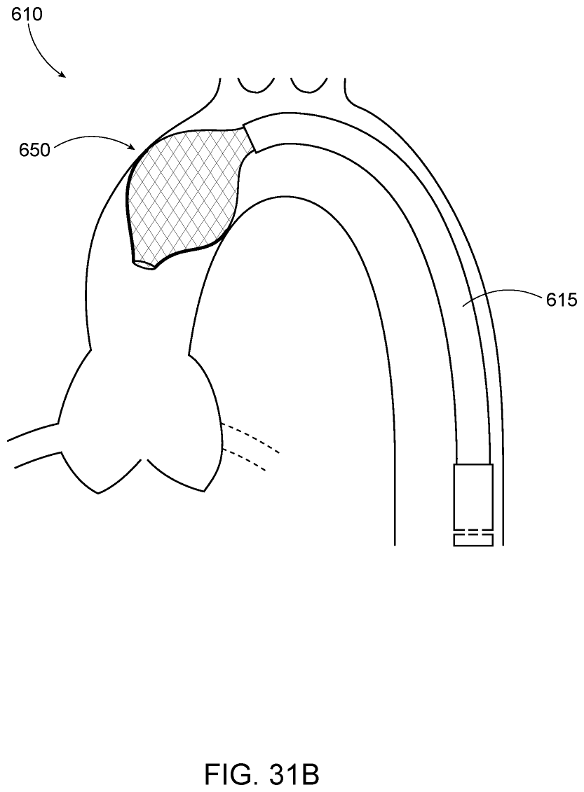

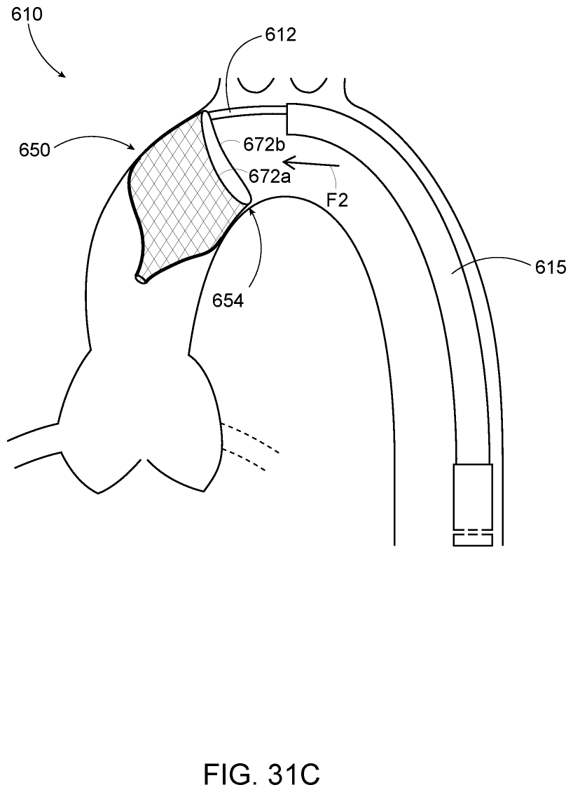

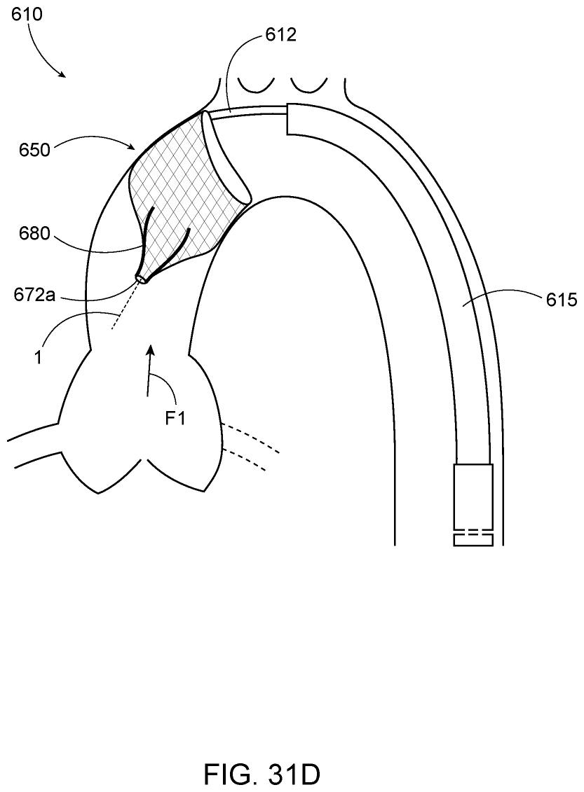

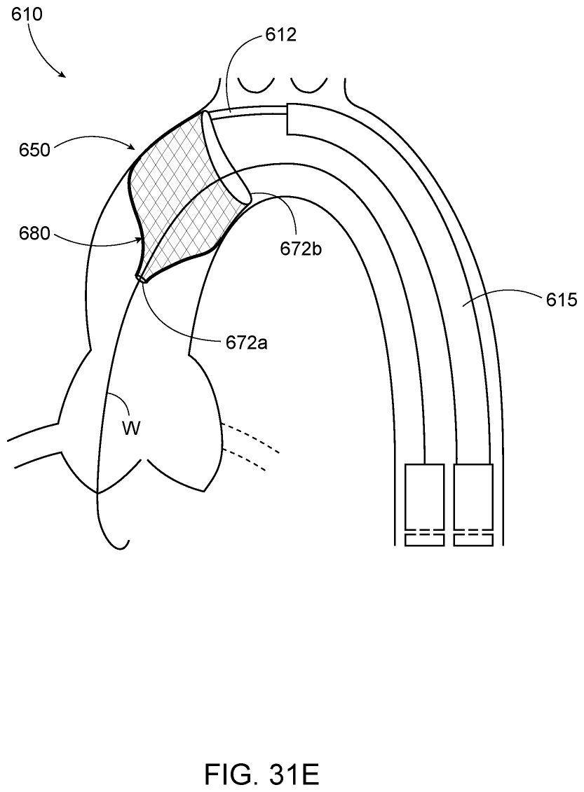

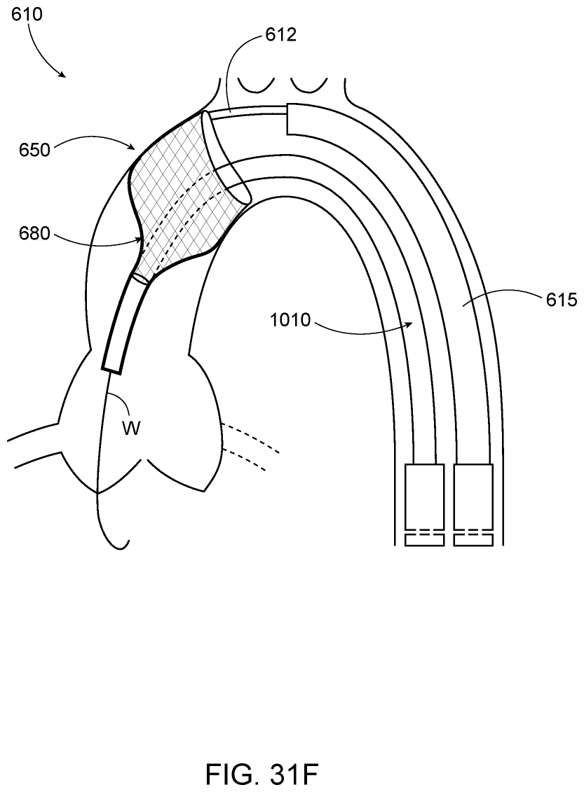

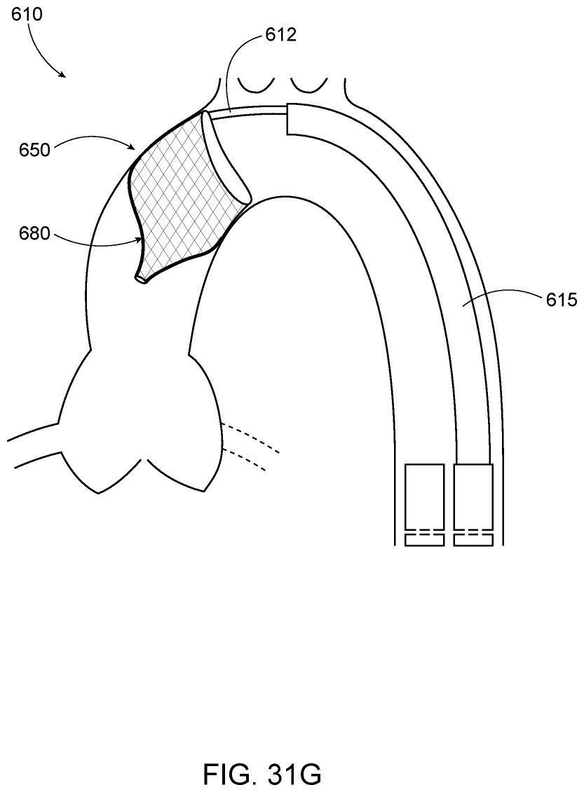

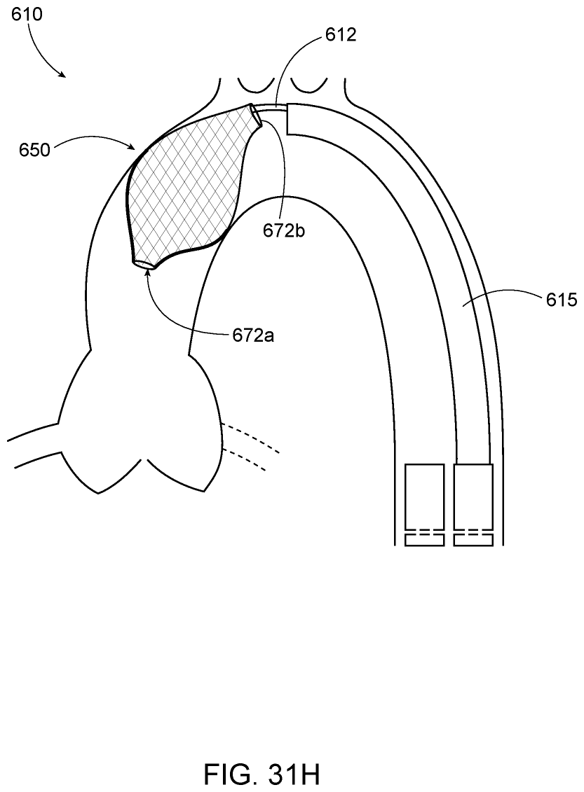

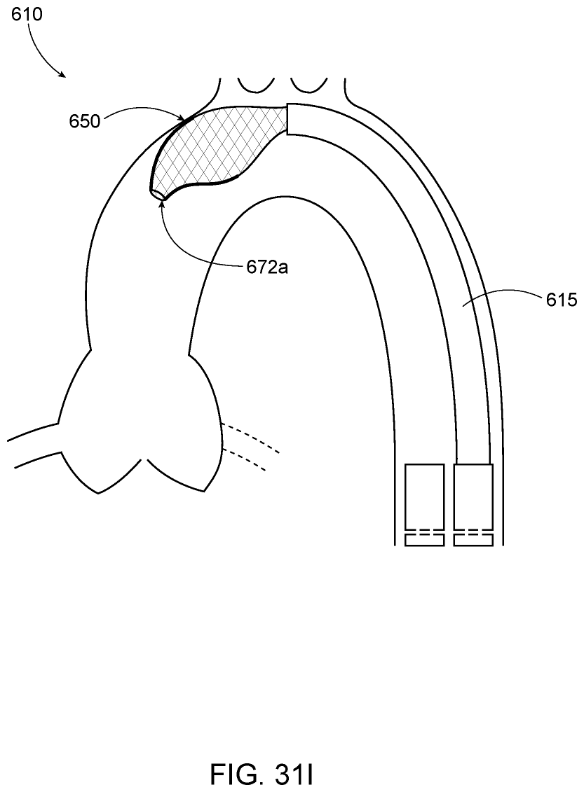

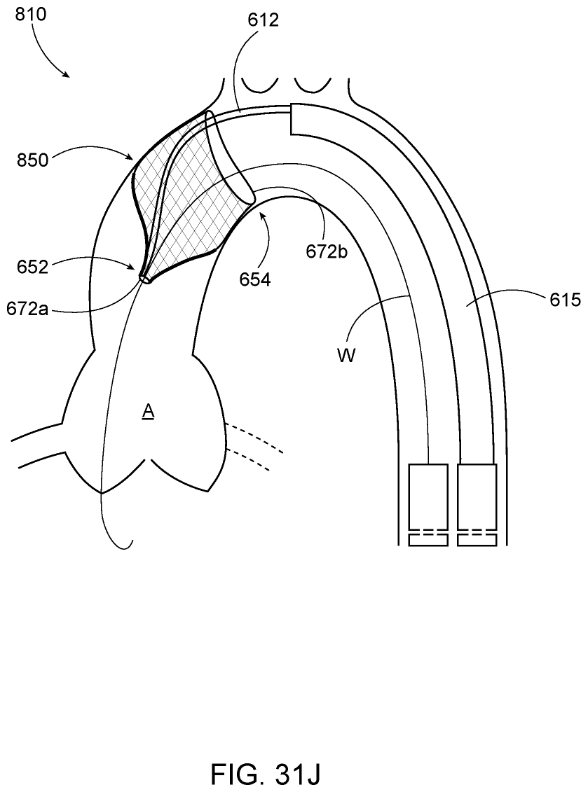

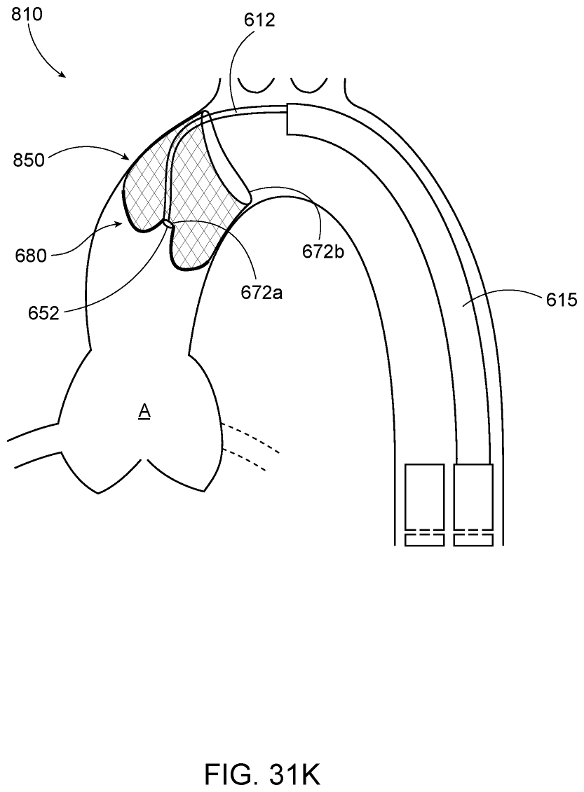

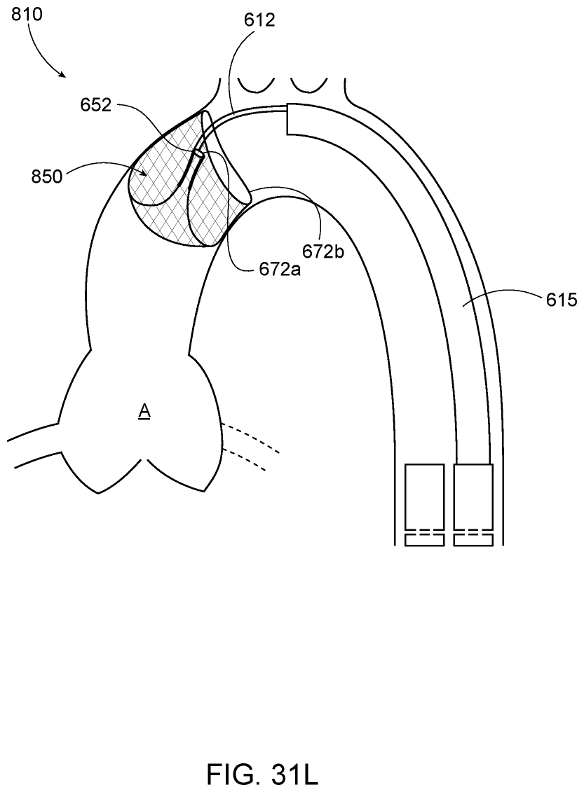

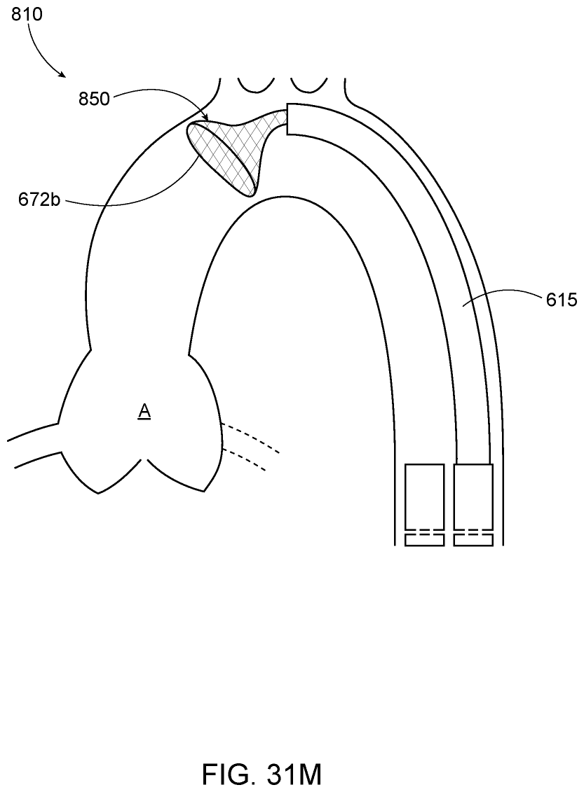

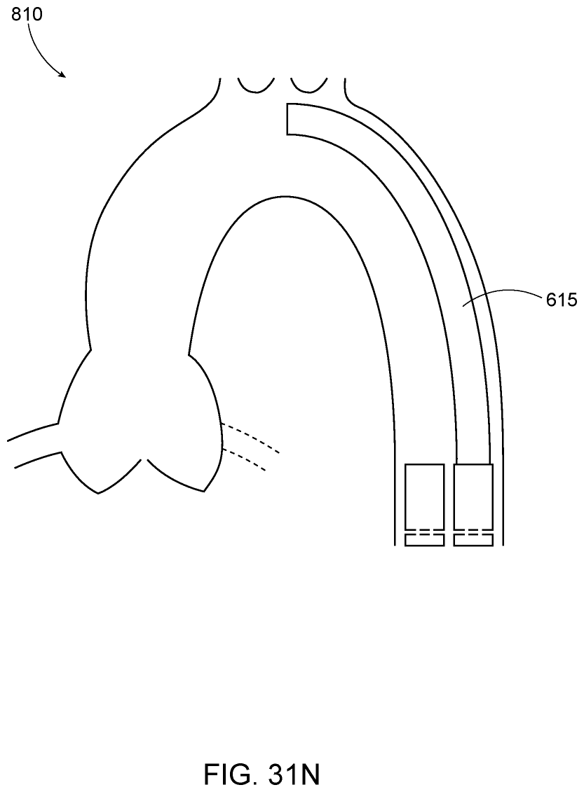

FIGS. 31A-31N illustrates an exemplary method for the procedure described above and incorporating the expandable shields 650,750 as described herein. As shown in FIG. 31A, the method may generally include placing a sheath 615 in the cardiovascular system, e.g. the ascending aorta. A guidewire(s) W may be inserted into the sheath 615 and its distal end positioned proximate or through the valve (native or implanted).

As shown in FIG. 31B, the expandable shield 650 is inserted into the ascending aorta and slidable over or along the guidewire W (not shown). The user may remove the guidewire W and allow the expandable shield 650 braid to unsheathe and expand upon exist from the sheath 615. The configuration of the shield 650 allows for auto expansion upon exit from the sheath 615. Next, as shown in FIGS. 31C and 31D, the expandable shield 650 is fully deployed. In FIG. 31C, the expandable shield 650 expands fully to contact the inner surface of the ascending aorta. The trailing end 654 and its loop 672b expand outwardly and may form the maximum cross-sectional dimension (perpendicular to the central axis 1, not shown) of the shield 650 in this state. The movable panels 680 are in the closed state when fluid flow is in the second direction F2, thereby preventing regurgitation flow to the left ventricle (LV). However, as shown in FIG. 31D, fluid flow in the first direction F1 collapses the movable panel 380 and causes a leading end 652 of the expandable shield 650 to contract toward the central axis 1.

Next, as shown in FIG. 31E, a wire W is advanced through the expandable shield 650, such as through tip and ring 672a. As shown in FIG. 31F, a surgical device 1010, for example an excision catheter as described above, is advanced through the expandable shield 650 to conform to an inner side of the shield 650. The surgical procedure, such as leaflet excision or another procedure, may then be performed as described above. This configuration allows the TAVR valve and delivery systems to pass through the center or tip of the expandable shield 650 as well. Next, as shown in FIGS. 31G through 31I, the user may remove all surgical devices and/or catheters. Using an actuator or other control device, a user may retract shaft 612 and cause loop 672b to collapse, thereby causing the expandable shield 650 to collapse toward the central axis 1. This allows the expandable shield 650 to be recaptured into the sheath 615. The surgical system 610 can then be removed.

An alternate embodiment is shown in FIG. 31J through 31N. The surgical system 810 includes an expandable shield 850 that can be inverted into an inverted cone shape to capture debris. The expandable shield 850 includes similar features to expandable shield 650 and 750 and similar references numbers are used to identify features common among shields 650, 750 and 850. More specifically, in FIG. 31J, the expandable shield 850 is shown with the distal end of the shaft 612 coupled to the leading end 652 of the expandable shield 650. FIG. 31J illustrates the wire W present in the shield 850. In FIGS. 31K through 31N, retraction of the shaft 612 causes the leading end 652 to pull inward, inside the shield 850 and toward the trailing end 654 and loop 672b. In FIG. 31M, the shaft 612 is further retracted into the sheath 615, pulling the loop 672a into the sheath 615 while the loop 672 remain outside of the sheath 615 and expanded outwardly. This has the result of inverting the expandable shield 850, creating a cone or funnel shape structure formed by the frame of the shield. In this state, any debris is captured by pulling the expandable shield 850 inside the sheath 615, which can collect the debris into the sheath 615, as shown in FIG. 31N. The sheath 615 may then be removed.

It will be appreciated by those skilled in the art that various modifications and alterations of the present disclosure can be made without departing from the broad scope of the appended claims. Some of these have been discussed above and others will be apparent to those skilled in the art. The scope of the present disclosure is limited only by the claims.

1.-2. (canceled)

3. A surgical system, comprising:
a shaft that is elongated along a central axis;
an expandable shield carried by the shaft, the expandable shield having an insertion configuration, where the expandable shield is collapsed toward the central axis, and an expanded configuration, where the expandable shield is expanded outwardly away from the central axis; and
a movable panel coupled to the expandable shield, wherein the movable panel is configured to open in response to a fluid flow in a first direction that impinges the expandable shield, and close in response to fluid flow in a second direction that is opposite the first direction that impinges the expandable shield.

4. The surgical system according to claim 3, wherein the expandable shield has a non-porous section, a porous section, wherein the movable panel overlies the porous section.

5. The surgical system according to claim 4, wherein the movable panel is configured, such that, 1) fluid flow in the first direction opens the movable panel to permit fluid to flow through the porous section, and 2) fluid flow in the second direction that is opposite the first direction causes the movable panel to close to inhibit fluid flow through the porous section.

6. The surgical system according to claim 3, further comprising an actuatable panel coupled to the expandable shield, wherein the actuatable panel is configured to transition between A) a closed configuration that inhibits fluid flow through the expandable shield in both the first direction and the second direction that is opposite the first direction, and B) an open configuration that permits fluid flow through the expandable shield in both the first direction and the second direction; and
an actuator coupled to the actuatable panel and configured to cause the actuatable panel to transition between the closed configuration and the open configuration.

7. The surgical system of claim 6, further comprising an actuation rod coupled to the actuatable panel and the actuator, such that actuation of the actuator causes the actuation rod to move the actuatable panel between the closed configuration and the open configuration; and an alignment element coupled to the actuation rod and the actuatable panel.

8. The surgical system according to claim 3, wherein the movable panel is one of:
a tapered panel that overlies a porous section of the expandable shield; and
a curved panel that overlies the porous section of the expandable shield.

9. (canceled)

10. The surgical system according to claim 3, wherein the movable panel includes at least one slit that overlies a porous section of the expandable shield.

11. The surgical system according to claim 3, wherein the movable panel is a) configured to rotate into an open configuration or b) configured to slide into the open configuration.

12. (canceled)

13. The surgical system according to claim 3, wherein the expandable shield has a leading end defining a tapered tip and a trailing end that is proximal relative to the leading end, wherein the expandable shield is configured to transition between the insertion configuration, where the expandable shield is collapsed toward the central axis in response to the first direction of fluid flow, and the expanded configuration, where the trailing end that expands outwardly in response the second direction of fluid flow.

14. The surgical system according to claim 3, wherein the movable panel is a plurality of movable panels that overlie a plurality of porous sections, respectively, wherein the plurality of movable panels are independently responsive to fluid flow to open or close.

15. (canceled)

16. The surgical system according to claim 3, further comprising:
an elongated shaft that extends through a central portion of the expandable shield; and
a nose cone carried by the elongated shaft, wherein the nose cone is movable relative to the expandable shield in at least a distal direction.

17.-21. (canceled)

22. The surgical system according to claim 3, wherein the expandable shield has a leading end, a trailing end spaced rearward of the leading end, an internal side, an external side that opposes the internal side, and an expandable wire frame that defines the trailing end, such that, the trailing end is expandable outwardly away from the central axis to open the expandable shield into the expanded configuration,
wherein the movable panel is located at the leading end and coupled to the internal side of the expandable shield, such that fluid flow that impinges the movable panel causes a leading portion of the expandable shield to contract toward the central axis, thereby permitting fluid flow.

23. The surgical system according to claim 3, wherein the expandable shield has a leading end, a trailing end spaced rearward of the leading end, an internal side, an external side that opposes the internal side, and an expandable wire frame that defines the trailing end, such that, the trailing end is expandable outwardly away from the central axis to open the expandable shield into the expanded configuration,
wherein the movable panel is located at the leading end and coupled to the external side of the expandable shield, such that fluid flow that impinges the movable panel causes a leading portion of the expandable shield to contract toward the central axis, thereby permitting fluid flow.

24. (canceled)

25. The surgical system according to claim 3, further comprising at least one deployment actuator coupled the expandable shield, the at least one deployment actuator configured to cause the expandable shield to transition between the insertion configuration and the expanded configuration.

26. The surgical system according to claim 3, wherein the expandable shield has a frame that is configured to expand outwardly away from the central axis.

27. The surgical system of claim 26, wherein the frame defines a funnel shape in the expanded configuration.

28. The surgical system of claim 26, wherein the frame has a proximal portion coupled to the shaft, a distal portion opposite and distal to the proximal portion, and at least one support bar that couples the proximal portion to the distal portion, wherein the distal portion expands outwardly away to the central axis while proximal portion does not expand outwardly.

29. The surgical system according to claim 26, wherein the frame includes a first wire defining a first portion of a leading end of the expandable shield and a second wire defining a second portion of the leading end of the expandable shield, and a plurality of support wires coupled to the first wire and the second wire, such that the first wire and second wire expand radially outwardly into a ring shape.

30. The surgical system according to claim 26, wherein the frame is a self-expanding wire mesh having a distal portion that expands radially outwardly and a proximal portion at a distal end of the shaft that remains collapsed toward the central axis.

31. The surgical system according to claim 26, wherein the frame includes a plurality of separate flexible arms, wherein each arm defines a terminal end such that the terminal ends of the separate flexible arms expand outwardly away from the central axis.

32. The surgical system according to claim 3, further comprising a sheath configured for placement in an aorta of a cardiovascular system, the sheath having a distal end, a proximal end, and a sheath channel that extends from the distal end to the proximal end.

33.-35. (canceled)
